(19) United States
(12) Patent Application Publication
(10) Pub. No.: US 2009/0312373 A1
(43) Pub. Date: Dec. 17, 2009

(54) METHODS FOR THE TREATMENT OF CANCER USING PIPERLONGUMINE AND PIPERLONGUMINE ANALOGS

(75) Inventors: Sam W. Lee, Newton, MA (US); Anna Mandinova, Newton, MA (US)

Correspondence Address: WOLF GREENFIELD & SACKS, P.C., 600 ATLANTIC AVENUE, BOSTON, MA, 02210-2206, US

(73) Assignee: The General Hospital Corporation, Boston, MA (US)

(21) Appl. No.: 12/401,099

(22) Filed: Mar. 10, 2009

Related U.S. Application Data
(60) Provisional application No. 61/069,004, filed on Mar. 11, 2008.
(60) Provisional application No. 61/055,318, filed on May 22, 2008.

(51) Int. Cl. A61K 31/44; A61P 35/00; A61P 9/00

(52) U.S. Cl. 514/350

(57) ABSTRACT

The invention provides methods for the treatment of cancer in a subject using piperlongumine and/or piperlongumine analogs.

### RELATED APPLICATIONS

This application claims the benefit under 35 U.S.C. 119(e) from U.S. provisional application Ser. No. 61/069,004 entitled “Methods for the treatment of cancer using piperlongumine and piperlongumine analogs” filed Mar. 11, 2008, and Ser. No. 61/055,318, entitled “Methods for the treatment of cancer using piperlongumine and piperlongumine analogs”, filed May 22, 2008, the entire contents of each of which are herein incorporated by reference.

### FIELD OF THE INVENTION

The invention provides methods for the treatment of cancer in a subject using piperlongumine and/or piperlongumine analogs.

### BACKGROUND OF THE INVENTION

The process of apoptosis, or programmed cell death is a physiological mechanism found in virtually all tissues (Hanahan, J. G., and Weinberg, R. A. (2000) The hallmarks of cancer. Cell 100, 57-70). Many normally developing tissues eliminate improperly developed cells by triggering their apoptotic cell death (Hanahan and Weinberg, 2000). However, in cancer cells this tightly regulated program is often deregulated and activation of cell survival signal transduction pathways can cause the cells to inappropriately survive, grow and divide (Vogelstein, B., and Kinzler, K. W. (2004) Cancer genes and the pathways they control. Nat. Med. 10, 789-799). This aberrant cellular behavior is the major hallmark of tumor growth. As such, drugs that stimulate apoptosis of cancer cells and therefore restore this normal cellular function can prevent the accumulation of tumor cells and lead to tumor regression. In addition, most aggressive tumors are very resistant to apoptosis induced by chemotherapeutic agents or radiation because of their impaired ability to undergo apoptosis as a result of genetic defects in the normal apoptosis pathways (Vogelstein and Kinzler, 2004).

Loss of p53 pathway function occurs commonly in human tumors and can contribute not only to aggressive tumor behavior but also to therapeutic resistance (Vogelstein and Kinzler, 2004). The p53 protein is a major target for mutational inactivation in human cancer and represents a major difference between normal cells and cancer cells. Therefore, current and future efforts toward developing new therapies to improve survival and quality of life of patients with these aggressive tumors must also include strategies that specifically target cancer cell resistance to apoptosis.

### SUMMARY OF THE INVENTION

In one aspect, the invention provides methods for the treatment of cancer in a subject. In some embodiments, the method for treating a cancer in a subject comprises administering to a subject in need of such treatment a therapeutically effective amount of a composition comprising piperlongumine and/or a piperlongumine analog to treat the cancer in the subject. In some embodiments, the administration of piperlongumine or a piperlongumine analog provides the administration of an effective dose of an anti-cancer compound with a low toxicity. In some embodiments, only a low dose of piperlongumine and/or a piperlongumine analog needs to be administered to be therapeutically effective because piperlongumine or a piperlongumine analog can “trigger” the suppression of cancer growth or the killing of cancer cells and is not required to maintain contact with the cancer cell to suppress cancer growth or kill the cancer cell.

In one aspect the invention provides methods for suppressing the accumulation of DNA damage in a normal cell (i.e., a non-cancer cell) by contacting the cell with a composition comprising piperlongumine and/or a piperlongumine analog. It was unexpectedly found that piperlongumine and/or a piperlongumine analog can suppress the accumulation of DNA damage in normal cells while inducing the accumulation of DNA damage in cancer cells. Thus, normal cells can be protected form the deleterious effects of increased levels of DNA damage by contacting the cells with piperlongumine and/or a piperlongumine analog. In some embodiments, a subject undergoing anti-cancer chemotherapy can be administered a therapeutically effective amount of a composition comprising piperlongumine and/or a piperlongumine analog thereby protecting the normal cells of the subject from DNA damage induced by anti-cancer chemotherapy. In some embodiments, a subject undergoing anti-cancer chemotherapy can be administered a therapeutically effective amount of a composition piperlongumine and/or a piperlongumine analog thereby increasing the accumulation of DNA damage in cancer cells in the subject. In some embodiments, the DNA damage is increased preferentially in cancer cells.

In one aspect, the method for treating a cancer in a subject comprises administering to a subject in need of such treatment a therapeutically effective amount of a composition comprising piperlongumine and/or a piperlongumine analog to treat the cancer in the subject. In some embodiments, the treatment inhibits further growth of the cancer. In some embodiments, the treatment results in regression of the cancer. In some embodiments, the cancer is a carcinoma, a sarcoma or a melanoma. In some embodiments, the piperlongumine analog comprises a piperlongumine conformation. In some embodiments, the piperlongumine analog is a piperlongumine compound in which one or more methoxy groups are replaced with a hydroxy group. In some embodiments, the piperlongumine analog is p-demethylated piperlongumine. In some embodiments, the effective amount is less than 50 mg/kg of piperlongumine or piperlongumine analog. In some embodiments, the effective amount is less than 10 mg/kg of piperlongumine or piperlongumine analog. In some embodiments, the effective amount is less than 1.5 mg/kg of piperlongumine or piperlongumine analog. In some embodiments, the effective amount is less than the oral LD50 in mouse. In some embodiments, the effective amount is less than the 10% of the oral LD50 in mouse. In some embodiments, the effective amount is less than the 1% of the oral LD50 in mouse. In some embodiments, the subject is otherwise free of symptoms treatable by piperlongumine or piperlongumine analog. In some embodiments, the method further comprises administering to the subject a non-piperlongumine anti-cancer compound. In some embodiments, the cancer is resistant to standard chemotherapies or anti-cancer compounds. In some embodiments, the growth of non-cancer cells that grow at a rate similar to the cells of the cancer is not significantly suppressed.

In another aspect, the invention provides a method for reducing angiogenesis in a subject. In some embodiments, the method for reducing angiogenesis in a subject comprises administering to a subject in need of such treatment a therapeutically effective amount of a composition comprising piperlongumine and/or a piperlongumine analog to reduce angiogenesis in the subject. In some embodiments, the treatment inhibits growth of a tumor. In some embodiments, the treatment results in regression of a tumor. In some embodiments, the tumor is a carcinoma or a sarcoma. In some embodiments, the piperlongumine analog comprises a piperlongumine conformation. In some embodiments, the piperlongumine analog is a piperlongumine compound in which one or more methoxy groups are replaced with a hydroxy group. In some embodiments, the piperlongumine analog is p-demethylated piperlongumine. In some embodiments, the effective amount is less than 50 mg/kg of piperlongumine or piperlongumine analog. In some embodiments, the effective amount is less than 10 mg/kg of piperlongumine or piperlongumine analog. In some embodiments, the effective amount is less than 1.5 mg/kg of piperlongumine or piperlongumine analog. In some embodiments, the effective amount is less than the oral LD50 in mouse. In some embodiments, the effective amount is less than the 10% of the oral LD50 in mouse. In some embodiments, the effective amount is less than the 1% of the oral LD50 in mouse. In some embodiments, the subject is otherwise free of symptoms treatable by piperlongumine or piperlongumine analog. In some embodiments, the method further comprises administering to the subject a non-piperlongumine anti-cancer compound.

In another aspect, the invention provides a method for inhibiting cell proliferation. In some embodiments, the method for inhibiting cell proliferation comprises contacting a cell with an effective amount of a composition comprising piperlongumine and/or a piperlongumine analog to inhibit the proliferation of the cell. In some embodiments, the method further comprises contacting the cells with a non-piperlongumine anti-cancer compound.

In another aspect, the invention provides a method for reducing metastasis and/or invasion of a cancer in a subject. In some embodiments, the method for reducing metastasis and/or invasion of a cancer in a subject comprises treating the subject with an effective amount of a composition comprising piperlongumine and/or a piperlongumine analog to reduce metastasis and/or invasion. In some embodiments, the method further comprises contacting the cells with a non-piperlongumine anti-cancer compound.

In another aspect, the invention provides a method for increasing apoptosis of a cell or in a population of cells. In some embodiments, the method for increasing apoptosis of a cell or in a population of cells, the method comprises contacting the cell or population of cells with an effective amount of a composition comprising piperlongumine and/or a piperlongumine analog to increase apoptosis in the cell or population of cells. In some embodiments, the number of apoptotic cells in a population of cells is increased by at least two-fold. In some embodiments, the number of apoptotic cells in a population of cells is increased by at least five-fold. In some embodiments, the number of apoptotic cells in a population of cells is increased by at least ten-fold. In some embodiments, the method further comprises contacting the cells with a non-piperlongumine anti-cancer compound. In some embodiments, the cell or population of cells is a cancer cell or population of cancer cells. In some embodiments, the cell or population of cells is in a subject.

In another aspect, the invention provides a method for increasing p53 activity in a cell or population of cells. In some embodiments, the method for increasing p53 activity in a cell or population of cells comprises contacting the cell or population of cells with an effective amount of a composition comprising piperlongumine and/or a piperlongumine analog to increase p53 activity in the cell or population of cells. In some embodiments, p53 activity is increased by the induction of p53 expression. In some embodiments, p53 activity is increased by the induction of p53 acetylation. In some embodiments, the method further comprises contacting the cells with a non-piperlongumine anti-cancer compound. In some embodiments, the cell or population of cells is a cancer cell or population of cancer cells. In some embodiments, the cell or population of cells is in a subject.

In another aspect, the invention provides a method for inducing DNA damage in a cancer cell or population of cancer cells, the method comprising contacting the cancer cell or population of cancer cells with an effective amount of a composition comprising piperlongumine and/or a piperlongumine analog to induce DNA damage in the cancer cell or population of cancer cells. In some embodiments, the cancer cell or population of cancer cells is in a subject.

In another aspect, the invention provides a method for preferentially inducing DNA damage in a cancer cell or population of cancer cells, the method comprising contacting the cancer cell or population of cancer cells with an effective amount of a composition comprising piperlongumine and/or a piperlongumine analog to induce DNA damage in the cancer cell or population of cancer cells, wherein the cancer cell or population of cancer cells is in a mixed population of cancer cells and normal cells. In some embodiments, the mixed population of cancer cells and normal cells is in a subject.

In another aspect, the invention provides a method for suppressing DNA damage in a cell or population of cells, the method comprising contacting the cell or population of cells with an effective amount of a composition comprising piperlongumine and/or a piperlongumine analog to suppress DNA damage in the cell or population of cells. In some embodiments, the cell or population of cells has been contacted with an anti-cancer compound. In some embodiments, the method further comprises contacting the cell or population of cells with an anti-cancer compound. In some embodiments, the cell or population of cells is in a subject.

In another aspect, the invention provides a pharmaceutical composition comprising piperlongumine and/or a piperlongumine analog and a pharmaceutically acceptable carrier. In some embodiments, the piperlongumine analog comprises a piperlongumine conformation. In some embodiments, the effective amount is less than 50 mg/kg of piperlongumine or piperlongumine analog. In some embodiments, the effective amount is less than 10 mg/kg of piperlongumine or piperlongumine analog. In some embodiments, the piperlongumine analog is a piperlongumine analog that has one or more methoxy groups replaced with a hydroxy group. In some embodiments, the piperlongumine analog is p-demethylated piperlongumine. In some embodiments, the effective amount is less than 1.5 mg/kg of piperlongumine or piperlongumine analog. In some embodiments, the effective amount is less than the oral LD50 in mouse. In some embodiments, the effective amount is less than the 10% of the oral LD50 in mouse. In some embodiments, the effective amount is less than the 1% of the oral LD50 in mouse. In some embodiments, the method further comprises administering to the subject a non-piperlongumine anti-cancer compound.

In another aspect, the invention provides a kit comprising a pharmaceutical composition comprising a therapeutically effective amount of piperlongumine and/or a piperlongumine analog, and instructions for preparation and/or administration of the pharmaceutical composition. In some embodiments, the effective amount is less than 50 mg/kg of piperlongumine or piperlongumine analog. In some embodiments, the effective amount is less than 10 mg/kg of piperlongumine or piperlongumine analog. In some embodiments, the effective amount is less than 1.5 mg/kg of piperlongumine or piperlongumine analog. In some embodiments, the effective amount is less than the oral LD50 in mouse. In some embodiments, the effective amount is less than the 10% of the oral LD50 in mouse. In some embodiments, the effective amount is less than the 1% of the oral LD50 in mouse. In some embodiments, the kit further comprises a pharmaceutically acceptable carrier. In some embodiments, the kit further comprises one or more non-piperlongumine anti-cancer compounds.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 shows an overview of the screening method for chemical activators of CDIP (Cell Death involved p53 target).

FIG. 2 shows the structure of piperlongumine, N-(3,4,5-trimethoxycinnamoyl)-Δ³-piperidine-2-one, an amide alkaloid (C₁₇H₁₉N0₅).

FIG. 3 shows that piperlongumine treatment stimulates luciferase activity of CDIP promoter containing p53 binding site in U2OS cells.

FIG. 4 shows that piperlongumine treatment activates a proapoptotic target, Puma, in human cancer cells regardless of p53 status (A & B); piperlongumine activates p53 in wt-p53 containing cancer cells (A).

FIG. 5 shows the anti-cancer selectivity of piperlongumine in human cancer cells. Piperlongumine treatment induces cell death in EJ human bladder cancer cells (A) and in HCT116 human colon cancer cells (B). Etoposide, a genotoxic agent, was used as control. FIG. 5(C) shows cell viable staining after piperlongumine treatment in EJ, HCT116 and U2OS cells. FIG. 5(D) shows that the sub-G1 apoptotic population of cells is increased by piperlongumine treatment in HCT116 cells.

FIG. 6 shows the inhibition of tumor growth by piperlongumine (CT-007) in human bladder tumor mice (A and B).

FIG. 7 shows the anti-tumor activity of piperlongumine (CT-007) in bladder and breast tumor in mice.

FIG. 8 shows the anti-tumor activity of piperlongumine (CT-007) in a lung tumor model.

FIG. 9 shows the anti-angiogenic effect of piperlongumine (CT-007).

FIG. 10 shows the staining of apoptosis gene expression in piperlongumine (CT-007) treated tumor mice.

FIG. 11 shows the anti-tumor effect of piperlongumine (CT-007) on a B16/F10 mouse melanoma model (A-C).

FIG. 12 shows the fold of repression of selected genes upon exposure of human cells with piperlongumine, as evaluated by Exon-array analysis (A: U2OS; B: EJ cells).

FIG. 13 shows that exposure to increased concentrations of piperlongumine (CT-007) results in increased suppression of survival gene expression in human cancer cells (A: U2OS; B: EJ cells).

FIG. 14 shows compounds related to piperlongumine.

FIG. 15 shows that piperlongumine (SP) induces p53 acetylation.

FIG. 16 shows that piperlongumine (SP2007) inhibits cell growth in human melanoma and ovarian cancer cell lines.

FIG. 17 shows that piperlongumine (SP2007) inhibits cell growth in human renal cancer cell lines.

FIG. 18 shows that piperlongumine (SP2007) inhibits cell growth in glioblastoma cell lines.

FIG. 19 shows that piperlongumine (SP2007) inhibits cell growth in a control cancer cell line and in drug resistant A549 human non-small lung carcinoma cell lines.

FIG. 20 shows that piperlongumine (SP2007) induces cell death/apoptosis in transformed cells (EJ, HCT116), but not in normal cells (fibroblasts, keratinocytes).

FIG. 21 shows the differential miRNA profile of both p53 wild type and p53 mutant cells upon piperlongumine (SP2007) treatment.

FIG. 22 shows that piperlongumine (SP2007) changes the induction of miRNA-10b, a Twist target gene which regulates metastasis and migration.

FIG. 23 shows that piperlongumine (piper) inhibits Twist expression in U2OS and EJ cells.

FIG. 24 shows that piperlongumine (piper) induces CDIP protein expression in HCT116 cells.

FIG. 25 shows that piperlongumine (SP2007) inhibits growth of patient-derived breast cancer tumor samples.

FIG. 26 shows that piperlongumine (SP2007) inhibits growth of patient-derived colon cancer tumor samples.

FIG. 27 shows that piperlongumine (SP2007) inhibits growth of patient-derived osteocarcoma samples.

FIG. 28 shows that piperlongumine (SP2007) inhibits tumor progression and angiogenesis.

FIG. 29 shows that piperlongumine (SP2007) dissociates the vimentin/p120ctn/N-cadherin complex and prevents cell migration, invasion and metastasis.

FIG. 30 shows that piperlongumine (SP2007) shows no toxicity in vital organs of the mouse.

FIG. 31 shows that piperlongumine (SP2007) inhibits tumor growth in a spontaneous mouse model of breast cancer (MMTV-PyVT).

FIG. 32 shows that piperlongumine (SP2007) inhibits multiple tumor growth in a spontaneous tumor model.

FIG. 33 shows control tissue and piperlongumine (SP2007) treated MMTV-PyVT mammary tumor tissue.

FIG. 34 shows non-limiting examples of piperlongumine analogs.

FIG. 35 shows that the piperlongumine analog p-demethylated piperlongumine inhibits cancer cell proliferation (EJ and U2OS cells).

FIG. 36 shows that the piperlongumine analog p-demethylated piperlongumine induces expression of PUMA and p53 (EJ and U2OS cells).

FIG. 37 shows mammary tumor growth inhibition by piperlongumine (SP2007) or Taxol treatment in breast transgenic tumor mice.

FIG. 38 shows that piperlongumine (SP2007) treatment induces CDIP in U2OS human cancer cells containing wt-p53.

FIG. 39 shows that piperlongumine (SP2007) inhibits expression of Twist and N-cadherin in cancer cells. A: scheme for SP2007-mediated repression of Twist expression and its downstream targets that are involved in tumor invasion/metastasis; B: SP2007 inhibits expression of Twist and its targets N-cadherin and p120 catenin in EJ and U2OS human cancer cells; C: SP2007 treatment inhibits Twist expression in MMTV-PyVT mammary tumor mice; D: SP2007 treatment inhibits N-cadherin expression in MMTV-PyVT mammary tumor mice.

FIG. 40 shows that piperlongumine (SP2007) treatment disrupts the p120-ctn complex with vimentin in EJ cancer cells. A: scheme for SP2007-mediated repression of Twist expression and its downstream targets that are involved in tumor invasion/metastasis; B: SP2007 treatment disrupts the p120-ctn complex with vimentin in EJ cancer cells.

FIG. 41 shows that piperlongumine (piper/SP2007) induces DNA damage selectively in cancer cells but not in normal human epithelial cells; A: SP2007 does not induce phosphorylated gamma-H2AX, p53 and p21 in normal human breast epithelial cells; B. SP2007 does not induce phosphorylated gamma-H2AX and p53 in immortalized human breast epithelial cells; C. SP2007 induces DNA damage (phosphorylated gamma-H2AX levels) in EJ bladder carcinoma and U2OS osteosarcoma cell lines.

FIG. 42 shows the persisting effects of piperlongumine (P10/P20) after the compound is removed compared to taxol (T10 and T20) and vehicle (DMSO).

FIG. 43 shows the plasma concentration-time curve of piperlongumine in C57BL/6 Mice following intravenous (iv) and oral (op) administration (mean±SD, n=3).

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

In some aspects, the invention provides methods for the treatment of cancer in a subject through the administration to a subject in need of such treatment a therapeutically effective amount of piperlongumine and/or a piperlongumine analog. In some aspects, the invention provides regimens for the treatment of cancer by administering piperlongumine and/or a piperlongumine analog at doses that are non-toxic to the subject.

It was surprisingly found, as shown in the experimental part below, that administering a low dose of piperlongumine was effective in the treatment of cancer. While the cytotoxic activity of piperlongumine was known, anti-tumor activity has only been observed when the amount of piperlongumine administered intraperitonally was as high as the oral LD50 (Bezerra et al. 2006, Br. J. of Medicine and Biol Res 39: 801-807). Thus, prior to the current disclosure, piperlongumine could not be used as an effective anti-cancer agent because of its high toxicity.

In one aspect, the invention provides methods for increasing apoptosis in a cell or population of cells by contacting the cell or population of cells with piperlongumine and/or a piperlongumine analog. It was surprisingly found, as shown in the experimental part below, that piperlongumine has a strong apoptotic activity, which had not been observed previously. Furthermore, the treatment methods of the current disclosure allow for the induction of apoptosis of cancerous cells in subject at doses that are non-toxic to the subject. Thus, in one embodiment, the invention provides a method for treating cancer in a subject through the induction of apoptosis of the cancerous cells in the subject. In one embodiment, the invention provides a method for treating cancer in a subject through the induction of apoptosis and necrosis of the cancerous cells in the subject.

In one aspect, the invention provides a method for reducing metastasis of cancer in a subject. In some embodiments, the method for reducing metastasis of cancer in a subject comprises treating the subject with an effective amount of a composition comprising piperlongumine and/or a piperlongumine analog to reduce metastasis of cancer in a subject. In some embodiments, the method for reducing metastasis in a subject comprises the suppression of Twist expression.

In one aspect the invention provides methods for the treatment of cancer cells that have a functional p53 (i.e., wt p53) and cancer cells that have a non-functional p53 (i.e., a mutated version of p53). Furthermore, it was surprisingly found that treatment with piperlongumine and/or a piperlongumine analog results in the induction of expression of p53 and the induction of p53 acetylation.

In one aspect, the invention provides methods for suppressing the expression and/or activity of proteins encoded by survival genes in a cell or population of cells by contacting the cell or population of cells with piperlongumine and/or a piperlongumine analog. Survival genes suppressed by piperlongumine and/or a piperlongumine analog include Bc12, survivin and XIAP. Furthermore, the treatment methods of the current disclosure allow for the suppression of expression and/or activity of survival genes of cancerous cells in subject at doses that are non-toxic to the subject. Thus, in one embodiment, the invention provides a method of treating in cancer in a subject through the suppression of expression and/or activity of survival genes of the cancerous cells in the subject.

In one aspect, the invention provides methods for activating the CDIP gene (Cell Death Involved p53-target) in a cell or population of cells by contacting the cell or population of cells with piperlongumine and/or a piperlongumine analog. Furthermore, the treatment methods of the current disclosure allow for the activation of the CDIP gene of cancerous cells in subject at doses that are non-toxic to the subject. Thus, in one embodiment, the invention provides a method of treating cancer in a subject through the suppression of expression and/or activity of survival genes of the cancerous cells in the subject.

In one aspect, the invention provides methods for inducing DNA damage in a cancer cell or population of cancer cells by contacting the cancer cell or population of cancer cells with piperlongumine and/or a piperlongumine analog. In some embodiments, the induction of DNA damage in a cell can result in the death of the cell.

Furthermore, it was unexpectedly found that piperlongumine and/or a piperlongumine analog can preferentially induce DNA damage in a cancer cell when compared to normal (i.e., non-cancer) cells. Moreover, treatment with piperlongumine and/or a piperlongumine analog actually results in the suppression of DNA damage in normal cells. Thus, piperlongumine and/or piperlongumine analogs can protect normal cells from the deleterious effect of DNA damage, which for instance occurs if the cell is exposed to anti-cancer compounds, or if a subject in undergoing anti-cancer treatment.

### Piperlongumine and Piperlongumine Analogs

Piperlongumine is an amide alkaloid, that can be isolated from a variety of plants, including Piper aborescens, Piper tuberculatum and the roots of Piper longum L. The Indian medicinal plant Piper longum L, (family: piperaceae) grows and is cultivated in different parts of India and other southeast Asian countries and root extracts and preparations are widely used in various Indian system of medicine including its high reputation in Ayurvedic medicine for treatment of diseases of the respiratory tract including, cough, bronchitis, asthma etc; as counter-irritant and analgesic when applied locally for muscular pain and inflammation; as snuff in coma and drowsiness and internally as a carminative; as a sedative in insomnia and epilepsy; a general tonic and haematinic; as a cholagogue in obstruction of bile duct and gall bladder; as an emmenagogue and abortifacient; and for miscellaneous purposes as anthelmintic and in dysentery and leprosy (Chatterjee A, and Dutta, C P. (1967) Alkaloids of Piper longum Linn. I. Structure and synthesis of piperlongumine and piperlonguminine. Tetrahedron 23, 1769-1781); Yang, Y. C., Lee, S. G., Lee, H. K., Kim, M. K., Lee, S. H., and Lee, H. S. (2002) A piperidine amide extracted from Piper longum L. fruit shows activity against Aedes aegypti mosquito larvae. J Agric Food Chem. 50, 3765-3767; Lee, S. E., Park, B. S., Bayman, P., Baker, J. L., Choi, W. S., and Campbell, B. C. (2007) Suppression of ochratoxin biosynthesis by naturally occurring alkaloids. Food Addit Contam. 24, 391-397; Lin, Z., Liao, Y., Venkatasamy, R., Hider, R. C., and Soumyanath, A. (2007) Amides from Piper nigrum L. with dissimilar effects on melanocyte proliferation in-vitro. J Pharm Pharmacol. 59, 529-536.).

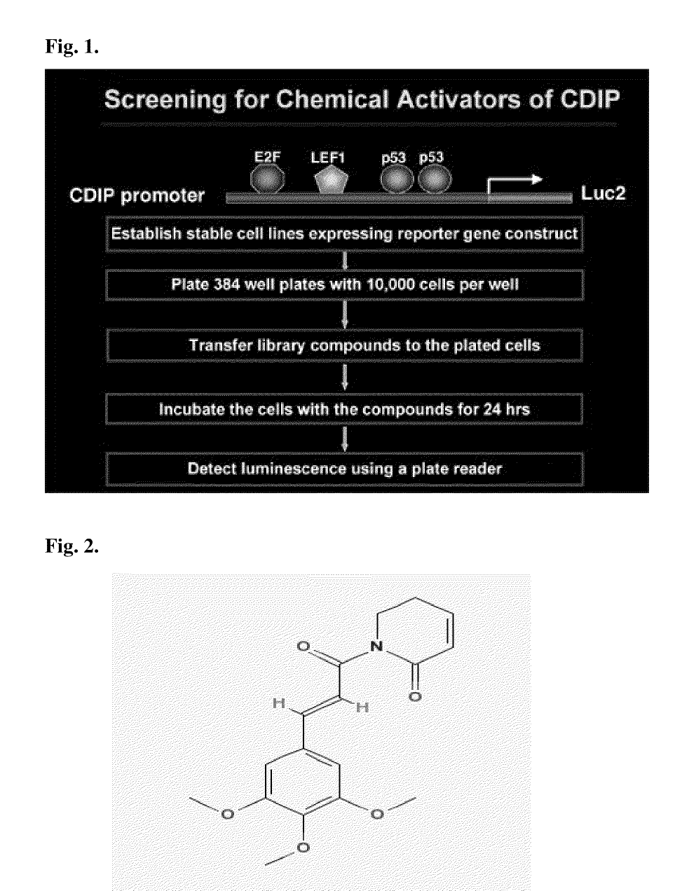

In addition to extraction piperlongumine from the roots of the Piper plant, piperlongumine can also be produced by organic synthesis (Chatterjee et al., 1967 Tetrahedron 23: 1769-1781). Piperlongumine, N-(3,4,5,-trimethoxycinnamoyl)-Δ³-piperidine-2-one, as used herein, is also called piplartine, SP, SP2007, piper and CT-007. The chemical structure of piperlongumine is shown in FIG. 2. The crystal structure of piperlongumine and the adopted conformation of the molecule are described by Banerjee et al. (Can J. Chem 1986, 64: 867-879).

Piperlongumine has been used to treat a variety of ailments, including asthma (Chatterjee et al., 1967 Tetrahedron 23: 1769-1781), depression (Ciecero et al., Phytomedicine, 2007, 14: 605-612) and blood disorders (Tsai et al., Plant Med 2005, 71: 535-542).

Piperlongumine analogs are chemically modified versions of piperlongumine that minimally comprise a piperlongumine conformation. In some embodiments, the piperlongumine analogs have one or more piperlongumine activities (as described herein), e.g., anti-cancer activity. The one or more activities are preferably present in the piperlongumine analog in significant amounts, e.g., at greater than 10%, 20%, 30%, 40%, 50%, 60%, 70%, 80%, or 90% of the activity of piperlongumine. More preferably, the one or more activities are preferably present in the piperlongumine analog at greater than 100%, 110%, 120%, 130%, 140%, 150%, 160%, 170%, 180%, 190%, 200%, or more, of the activity of piperlongumine. The piperlongumine analog may not have all of the activities of piperlongumine. However, non-active piperlongumine analogs, having none of the activities of piperlongumine in significant amounts, are not useful in the methods of the invention.

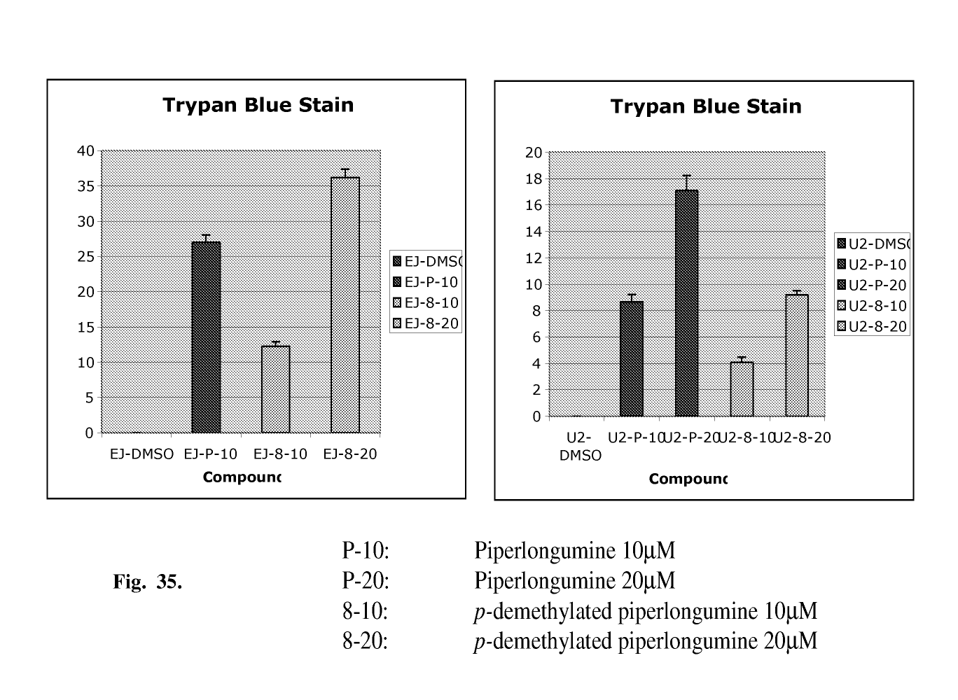

In some embodiments, piperlongumine analogs are piperlongumine analogs in which one or more of the methoxy groups have been modified or replaced (See e.g., FIG. 35; Duh et al. J. Nat. Prod. 1990 November-December, 53(6) 1575-1577; Duh et al., Phytochemistry 1990, 29: 2689-2691).

In some embodiments, piperlongumine or piperlongumine analogs are modified to improve bioavailability. In some embodiments, piperlongumine or piperlongumine analogs are modified to improve solubility. In some embodiments, one or more methoxy groups of piperlongumine or piperlongumine analogs have been replaced with a hydroxyl substituent. In some embodiments, the piperlongumine analog is demethylated, such as p-demethylated piperlongumine (XL-11-8), or other piperlongumine analogs wherein one or more methoxy groups has been replaced by a hydroxy group. In some embodiments, one or more methoxy groups of piperlongumine or piperlongumine analogs have been replaced with the substituents of the formula:

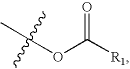

wherein R₁ is selected from the group consisting of —H, —CH₃, —(CH₂)ₙCH₃, —(CH₂)ₙCO₂H, —(CH₂)ₙN(C₁-C₅Alkyl)₂, —NH(CH₂)ₙN(C₁-C₅Alkyl)₂, —NHCHR₂CO₂H, and —NHCHR₂CO₂—(C₁-C₅Alkyl); wherein R₂ is a side chain selected from one of the twenty naturally-occurring amino acids; and wherein n=1-10.

The invention also embraces prodrugs of piperlongumine and piperlongumine analogs. Prodrugs of piperlongumine and piperlongumine analogs are modified versions of piperlongumine and piperlongumine analogs that may have improved stability and/or handling properties compared to the unmodified version of piperlongumine or piperlongumine analog. Prodrugs of piperlongumine and piperlongumine analogs are metabolized in vivo to result in piperlongumine and piperlongumine analogs, respectively.

The piperlongumine conformation is described by Banerjee et al. (Can J. Chem 1986, 64: 867-879), who show that the piperidone ring is in a distorted boat conformation, in contrast to the piperidine ring found in several amide alkoids isolated from the Piper species, which ring is found in the chair conformation. As shown in the experimental part below, when the piperidone ring of piperlongumine is modified, the modified compound (piperlongumine analog) loses its anti-cancer activity (See Example 7).

Piperlongumine and piperlongumine analogs are also referred to herein, collectively or individually, as the “compounds of the invention”.

### Treating a Cancer in a Subject

In one aspect, the invention provides methods for treating a cancer in a subject by administering to a subject in need of such treatment a therapeutically effective amount of a composition comprising piperlongumine and/or a piperlongumine analog to treat the cancer in the subject. As used herein, “treating a cancer” includes, but is not limited to, preventing the development of a cancer, reducing the symptoms of cancer, inhibiting the growth of an established cancer, preventing metastasis and/or invasion of an existing cancer, promoting or inducing regression of the cancer, inhibiting or suppressing the proliferation of cancerous cells, reducing angiogenesis or increasing the amount of apoptotic cancer cells. In some embodiments, the compounds of the invention are administered to a subject at risk of developing a cancer for the purpose of reducing the risk of developing the cancer.

In some embodiments, the compounds of the invention are selective for treatment of a specific cancer. In some embodiments, the compounds of the invention can be used to treat cancer comprising cancer cells with active p53 signaling pathways. In some embodiments, the compounds of the invention can be used to treat cancer comprising cancer cells with inactive p53 signaling pathways. In some embodiments, the compounds of the invention can be used to treat cancer comprising cancer cells that are resistant to apoptosis. In some embodiments, the compounds of the invention can be used to treat cancer comprising cancer cells that are resistant to the suppression of survival genes. In some embodiments, the compounds of the invention can be used to treat cancer comprising cancer cells that are susceptible to the suppression of survival genes.

In some embodiments, the compounds of the invention can be used to treat carcinomas, sarcomas, melanomas and hematopoietic cancers. In some embodiments, the compounds of the invention can be used to treat carcinomas, sarcomas or melanomas but not hematopoietic cancers. In some embodiments, the compounds of the invention can be used to treat carcinomas or melanomas, but not sarcomas or hematopoietic cancers. In some embodiments, the compounds of the invention can be used to treat sarcomas or melanomas, but not carcinomas or hematopoietic cancer. In some embodiments, the compounds of the invention can be used to treat carcinomas or sarcomas, but not melanomas or hematopoietic cancers. In some embodiments, the compounds of the invention can be used to treat sarcomas, but not carcinomas, melanomas or hematopoietic cancer. In some embodiments, the compounds of the invention can be used to treat carcinomas, but not sarcomas, melanomas or hematopoietic cancer. In some embodiments, the compounds of the invention can be used to treat melanomas, but not sarcomas, carcinomas or hematopoietic cancer.

In some embodiments, the compounds of the invention can be used to treat cancers that are resistant to treatment by standard chemotherapies and anti-cancer compounds. In some embodiments, the cancer is resistant to one or more non-piperlongumine anti-cancer compound provided herein.

In some embodiments, the compounds of the invention can be used to treat cancers that are resistant to treatment by piperine (an alkaloid amide related to piperlongumine).

In some embodiments, the compounds of the invention can be used to treat cancer in subjects with increased susceptibility to kidney toxicity.

In some embodiments, treatment with the compounds of the invention results in a statistically significant suppression of the growth of cancer cells but does not result in a statistically significant suppression of the growth of non-cancer cells. The terms “non-cancer cells”, “non-tumor cells”, “healthy cells” and “normal cells”, are used interchangeably herein, and refer to cells that are not undergoing the uncontrolled growth that characterizes cancer cells.

In some embodiments, the non-cancer cells grow at a rate that is similar to the growth rate of the cancer cells. A statistically significant suppression in the growth of treated cells is defined as greater than 5%, 10%, 20%, 30%, 40%, 50%, 60%, 70%, 80%, or 90% suppression of growth in comparison with untreated cells. A “growth at a rate similar to” is defined as a difference in growth rates between cell lines that is less than 1%, 5%, 10%, 20%, 30%, 40%, 50%, 60%, 70%, 80%, or 90%.

In some embodiments, piperlongumine and/or piperlongumine analogs “trigger” the suppression of growth of cancer cells. For instance, cancer cells can be exposed to piperlongumine or piperlongumine analogs only for a short period of time, and even after the piperlongumine or piperlongumine analog is removed, the anti-cancer effect is maintained. Thus, piperlongumine or the piperlongumine analog does not need to maintain contact with the cancer cell to suppress the growth of the cancer cell or to induce its killing. This “trigger” mechanism of action is in contrast to the mechanism of action of many other anti-cancer agents, such as taxol, that need to be in contact with the cancer cell to suppress the growth, or kill, the cancer cell. A “trigger” mechanism of action allows for the administration of lower doses of anti-cancer compound (e.g., piperlongumine or piperlongumine analog) than a mechanism of action wherein the anti-cancer compound needs to be in contact with the cancer cell to be effective to suppress the growth of the cancer cell or induce its killing.

While the invention is not limited to a specific mechanism of treating cancer in a subject, and/or killing cancer cells, by piperlongumine or a piperlongumine analog, it is likely that the accumulation of DNA damage within the cancer cell, which is induced by contacting the cancer cell with piperlongumine or a piperlongumine analog, results in the killing of the cancer cell.

p53

The role of p53 protein as the tumor suppressor in the response to cellular stresses has been extensively studied in the last decade (See e.g., Vousden, K. H., and Lu, X. (2002). Live or let die: the cell's response to p53. Nat Rev Cancer 2, 594-604.). As a component of the response to diverse acute stresses, p53 has a well-established role in a complex tumor suppressor network that mediates cellular responses to stress. p53 is activated in response to diverse cellular insults, including mitogenic oncogenes, hypoxia, oxidative stress, and DNA damage. Once activated, p53 can trigger a variety of anti-proliferative programs, including apoptosis, cellular senescence or cell cycle arrest, by targeting multiple components of each program's effector machineries. Since many of the chemotherapeutic agents currently used to treat cancer directly or indirectly damage DNA, they often rely on the integrity of the p53 pathway to elicit their anti-tumor effects. p53 functions as a transcription factor to regulate both positively and negatively the expression of a diverse group of responsive genes. These downstream genes play an important role in the early and late events as well as cross-talk between the extrinsic and intrinsic pathways of apoptosis.

Based on the observation that p53 function is lost in most cancers and its definitive role as the tumor suppressor it has long been thought that p53 would be an attractive target for new cancer therapies. However, it has not long been clear whether fixing a single gene could curb tumor growth or regression. Recently, using sophisticated mouse models, it has been demonstrated that restoration of p53 function in established tumors such as lymphomas, sarcomas and hepatocellular carcinomas, leads to regression of these tumors in vivo (Lowe, S. W., Cepero, E., and Evan, G. (2004) Intrinsic tumour suppression. Nature 432, 307-315; Fridman, J. S., and Lowe, S. W. (2003) Control of apoptosis by p53. Oncogene 22, 9030-9040). Thus, restoring p53 function in vivo in tumors represents an effective new approach to treating cancer.

The transcriptional activity of p53 is critical for growth inhibitory and apoptotic responses to a wide rage of insults. Through analysis of gene expression patterns, a number of p53 downstream target genes have been identified. Known transcriptional targets for p53 in promoting apoptosis includes various pro-apoptotic Bc12 members, including Puma, Noxa, Bid and Bax, as well as components of death-receptor signaling (e.g., DR5, Fas/CD95) and the apoptotic-effector machinery including KILLER/DR5, Bid, and Caspase 6 (Oren, M. (2003) Decision making by p53: life, death and cancer. Cell Death Differ. 10, 431-442; Benchimol, S. (2004) p53—an examination of sibling support in apoptosis control. Cancer Cell 6, 3-4; Benchimol, S. (2001) p53 dependent pathways of apoptosis. Cell Death Differ, 8, 1049-1051; Attardi, L. D., Reczek, E. E., Cosmas, C., Demicco, E. G., McCurrach, M. E., Lowe, S. W., and Jacks, T. (2000). PERP, an apoptosis-associated target of p53, is a novel member of the PMP-22/gas3 family. Genes Dev 14, 704-718; Hupp, T. R., Meek, D. W., Midgley, C. A., and Lane, D. P. (1992). Regulation of the specific DNA binding function of p53. Cell 71, 875-886; Nakano, K., and Vousden, K. H. (2001). PUMA, a novel proapoptotic gene, is induced by p53. Mol Cell 7, 683-694). Several pro-apoptotic genes including PUMA, Noxa, Perp, Bax, etc., have been identified as p53-target genes with p53 transcriptional response elements. The PUMA and Noxa genes are highly expressed in cells undergoing p53-dependent apoptosis, and their overexpression is sufficient to induce cell death, implicating these genes as important effectors of p53 pro-apoptotic function (Shibue, T., Suzuki, S., Okamoto, H., Yoshida, H., Ohba, Y., Takaoka, A., and Taniguchi, T. (2006) Differential contribution of Puma and Noxa in dual regulation of p53-mediated apoptotic pathways. EMBO J. 25, 4952-4962).

p53 pro-apoptotic targets are potent pro-apoptotic proteins and have a key function in the positive regulation of apoptosis in certain cancer cells. Because these p53 target proteins promote apoptosis, therapeutic strategies targeting these pro-apoptotic proteins are effective to overcome apoptosis resistance of certain cancer cells thereby developing a new class of cancer therapy to improve survival and quality of life of cancer patients. We recently identified a novel pro-apoptotic p53 target gene named CDIP (Cell Death Involved p53-target) (Brown, L., Ongusaha, P. P., Kim, H.-G., Nuti, S., Khosravi-Far, R., Aaronson, S. A. and Lee, S. W. CDIP, a novel p53 target gene, regulates TNFα-mediated apoptosis in a p53-dependent manner. EMBO J. 26: 3410-3422, 2007). CDIP itself potently induces cell death/apoptosis in human cancer cells regardless of p53 status. CDIP-dependent apoptosis is associated with caspase-8 activation. Furthermore, the CDIP-induced apoptosis is much more effective than other known p53 pro-apoptotic targets including PUMA or Noxa. Thus, that impaired CDIP induction in response to genotoxic stress or apoptotic stimuli, and the subsequent failure of consequent downstream signaling events leading to cell death, confers a survival advantage to tumor-prone cells, allowing them to escape apoptosis.

As shown below in the Examples, we screened for chemical compounds, e.g., small molecules, that are activators of CDIP. Piperlongumine was identified as such an activator of CDIP.

In some embodiments, the compounds of the invention can be used to treat cancer comprising cancer cells with active p53 signaling pathways. In some embodiments, the compounds of the invention can be used to treat cancer comprising cancer cells with inactive p53 signaling pathways. In some embodiments, treatment results in the increase in p53 activity in a cell or population of cells. An “increase in p53 activity”, as used herein, includes an increase of the activity of the p53 protein and may also include an increase of the activity of downstream targets of p53. The downstream targets of p53 can be activated by p53 protein or through any other mechanism.

p53 activity can be increased through a variety of mechanisms, which are all embraced by the invention. For instance, p53 activity can be increased by upregulating factors that stimulate p53, or by downregulating factors that inhibit or suppress p53 activity. In some embodiments, p53 activity is increased by activating CDIP. In some embodiments, p53 activity is increased by increasing the expression level of p53. In some embodiments, p53 activity is increased by increasing the acetylation level of p53. In some embodiments, p53 activity is increased by activating downstream targets of p53. In some embodiments, p53 activity is increased by increasing the expression level of the downstream targets of p53. In some embodiments, p53 activity is increased by modifying p53. In some embodiments, p53 activity is increased by modifying downstream targets of p53. Protein modifications are known in the art and include phosphorylation, proteolytic processing etc.

### DNA Damage

In one aspect, the invention provides methods for inducing DNA damage in a cancer cell or population of cancer cells by contacting the cancer cell or population of cancer cells with piperlongumine or a piperlongumine analog. DNA damage include both mutations of the DNA and compromising the integrity of the DNA, such as DNA strand breaks (both single stranded and double stranded). Furthermore, additional DNA damage can be generated when the cell tries to repair the mutated DNA or when mutated DNA is replicated. Thus, in one aspect the invention provides a method for inducing DNA damage in a cancer cell or population of cancer cells by contacting the cancer cell or population of cancer cells with an effective amount of a composition comprising piperlongumine and/or a piperlongumine analog.

Furthermore, it was unexpectedly found that piperlongumine and/or piperlongumine analogs can suppress the levels of DNA damage in normal cells. Thus, the invention also provides a method for suppressing DNA damage in a normal cell or population of normal cells by contacting the normal cell or population of normal cells with an effective amount of a composition comprising piperlongumine and/or a piperlongumine analog. Suppressing DNA damage in a normal cell or population of normal cells means decreasing the number of DNA damage in the normal cell or population of normal cells contacted with piperlongumine and/or a piperlongumine analog, compared to normal cells or a population of normal cells that are not contacted, by at least 5%, at least 10%, at least 20%, at least 30%, at least 40%, at least 50%, at least 60%, at least 70%, at least 80%, at least 90%, at least 100%, at least 2 times, at least 5 times, at least 10 times, at least 20 times, at least 50 times, at least 100, at least 1000 times, or more. In some embodiments, the composition comprising piperlongumine and/or a piperlongumine analog is administered when the subject is undergoing anti-cancer therapy, including the administration of anti-cancer compounds. In some embodiments, the composition comprising piperlongumine and/or a piperlongumine analog is administered when the subject is undergoing anti-cancer therapy, including the administration of anti-cancer compounds.

The invention also provides a method for preferentially inducing DNA damage in a cancer cell or population of cancer cells, by contacting the cancer cell or population of cancer cells with an effective amount of a composition comprising piperlongumine and/or a piperlongumine analog wherein the cancer cell or population of cancer cells is in a mixed population of cancer cells and normal cells, by contacting the cancer cell or population of cancer cells with an effective amount of a composition comprising piperlongumine and/or a piperlongumine. Preferentially inducing DNA damage in a cancer cell or population of cancer cells, wherein the cancer cell or population of cancer cells is in a mixed population of cancer cells and normal cells, means increasing the amount of DNA damage in the cancer cells compared to normal cells by at least 5%, at least 10%, at least 20%, at least 30%, at least 40%, at least 50%, at least 60%, at least 70%, at least 80%, at least 90%, at least 100%, at least 2 times, at least 5 times, at least 10 times, at least 20 times, at least 50 times, at least 100, at least 1000 times, or more.

### Subject

In one aspect, the invention provides methods for the treatment of cancer in a subject. A “subject”, as used herein, is a human or vertebrate mammal including, but not limited to, mouse, rat, dog, cat, horse, cow, pig, sheep, goat, or non-human primate. In some embodiments, the subject is otherwise free of symptoms treatable by piperlongumine or piperlongumine analogs. Symptoms treatable by piperlongumine or piperlongumine analogs include depression (Cicero et al., Phytomedicine, 2007, 14: 605-612), blood disorders (Tsai et al., Plant Med 2005, 71: 535-542) and asthma (Chatterjee et al., Tetrahedron 1967, 23: 1769-1781).

A “subject in need of treatment”, as used herein, means a subject that is identified as being in need of treatment. For instance, a subject in need of cancer treatment is a subject identified as having cancer or being at risk for developing cancer. A subject may be diagnosed as being in need of treatment by a healthcare professional and/or by performing one or more diagnostic assays. For instance, a subject in need of cancer treatment may be a subject diagnosed with cancer or being at risk of cancer by a healthcare professional. Diagnostic assays to evaluate if a subject has a cancer or is at risk for developing cancer are available in the routine art.

In some embodiments, the subject has a decreased tolerance level for the toxic effects of anti-cancer compounds, including the compounds of the invention. In some embodiments, the subject has a compromised kidney function.

### Angiogenesis

Piperlongumine also is demonstrated herein to have beneficial effects in angiogenesis. Thus, in one aspect, the invention provides a method for the reduction of angiogenesis in a subject by administering piperlongumine or a piperlongumine analog. By angiogenesis herein is meant a disease state which is marked by either an excess or an increased blood vessel development. Solid tumors typically require angiogenesis to support or sustain growth, e.g., breast, colon, lung, brain, bladder, and prostate tumors. Thus, reduction of angiogenesis provides a treatment methods for specific tumors. In some embodiments, the reduction of angiogenesis maybe concomitant with a decrease in tumor mass.

### Apoptosis

Piperlongumine also is demonstrated herein to have beneficial effects in apoptosis. Thus, in one aspect, the invention provides methods for increasing apoptosis in a cell or population of cells by contacting the cell or population with a therapeutically effective amount of a composition comprising piperlongumine and/or a piperlongumine analog. In some embodiments, the number of apoptotic cells in a population of cells is increased by at least 10%, 20%, 30%, 40%, 50%, 60%, 70%, 80%, 90% or 100%. In other embodiments, the number of apoptotic cells in a population of cells is increased by at least two-fold, three-fold, four-fold, or five-fold. In some embodiments, the number of apoptotic cells in a population of cells is increased by at least ten-fold.

An increase in apoptosis can be induced through a variety of mechanisms including but not limited to, activation of p53, induction of CDIP and suppression of survival gene function. In some embodiments, apoptosis is increased through the activation of p53. In some embodiments, apoptosis is increased through the induction of CDIP. In some embodiments, apoptosis is increased through the suppression of survival gene function.

In some embodiments, the increase of apoptosis will result in a decrease of the amount of cancer cells in a subject. Apoptosis refers to the process of programmed cell death. Apoptosis guides cell selection and regulation of cell population in the developing organism. In a mature organism, apoptosis additionally functions to rid the body of damaged or mutated cells. Cancerous cells which exhibit abnormal proliferation are thought to lack the ability to undergo appropriate apoptotic cell death. The process of apoptosis differs from simple necrosis which is a non-programmed form of cell death in response to injury. In some embodiments, the invention provides methods for increasing the number of cells in a cell population in, or undergoing, apoptosis, while not increasing the number of cells in, or undergoing, necrosis. Apoptosis can be measured by standard assays well known to those of skill in the art. Such assays include analysis of DNA ladder formation, TDT-mediated dUTP-biotin, nick end labeling (TUNEL), cell morphology, caspase-3 activation, etc.

### Inhibiting Cell Proliferation

Piperlongumine also inhibits cell proliferation. In one aspect, the invention provides methods for inhibiting cell proliferation by contacting the cell with a therapeutically effective amount of a composition comprising piperlongumine and/or a piperlongumine analog. Inhibiting cell proliferation can be achieved through a variety of mechanisms which are all embraced by the invention. For instance, cell proliferation can be inhibited by preventing DNA or protein synthesis, activating apoptotic or necrotic pathways, or reducing the amount or composition of nutrients available to a cell. In some embodiments, cells that have a higher potential to proliferate (e.g., cancer cells) are more strongly inhibited when compared to cells that have a lower potential to proliferate. In some embodiments, inhibiting cell proliferation according to the methods of the invention will result in the treatment of cancer in a subject.

### Metastasis and Invasion

Piperlongumine also suppresses metastasis and invasion of a cancer in a subject. In one aspect, the invention provides methods for reducing metastasis and/or invasion of a cancer in a subject by administering to a subject in need of such treatment a therapeutically effective amount of a composition comprising piperlongumine and/or a piperlongumine analog to reduce metastasis and/or invasion of the cancer in the subject. In some embodiments, piperlongumine and a piperlongumine analog is more effective in suppressing metastasis, invasion and the appearance of secondary or metastatic tumors, than well known anti-cancer compounds, such as taxol.

Metastatic cancers originate from a primary tumor. Metastasis of the primary tumor produces secondary tumors and disseminated cancer. It is well known that both primary and secondary tumors shed large numbers of cells. The shed cells can spread through the body. For instance, a primary tumor may damage the surrounding lymph or circulatory vessels, allowing entry of shed cells into the lymph or circulatory systems, and hastening their spread in the body. Moreover, shedding of cells by cancerous tumors increases during surgery and radiotherapy. For metastasis to occur the primary tumor physically must invade interstitial space of the primary tissue and penetrate the basement membrane of the tissue. Cancer cells that enter the lymph or blood must lodge at a new site in the circulatory system, extravasate out of the vessel into the interstitial space and invade the interstitial space of the secondary organ and proliferate in the new location.

Several enzyme systems have been implicated in the metastatic process including metalloproteinases, cysteine proteases, and serine proteases. The metastatic process also involves complex intracellular mechanisms that alter cancerous cells and their interactions with surrounding cells and tissues. One pathway that has been associated with the induction of metastasis and invasion is the expression of miR-10b by the transcription factor Twist, wherein the levels of miR-10b are correlated with metastasis and cell invasion. (Ma et al. Nature, 2007, 449: 682-688). Thus, the down-regulation of miR-10b can function as a marker for the suppression of metastasis In addition, a protein complex that is associated with the cell migration process and metastasis is the vimentin-cadherin-p120 catenin complex. Disassembly of this complex abrogates the ability of cells to metastasize (Hsu et al. Cancer Res. 2007, 22: 11064).

### Cancer

In one aspect, the invention provides methods for the treatment of cancer. “Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Carcinomas are malignant cancers that arise from epithelial cells and include adenocarcinoma and squamous cell carcinoma. Sarcomas are cancer of the connective or supportive tissue and include osteosarcoma, chondrosarcoma and gastrointestinal stromal tumor. Hematopoietic cancers, such as leukemia, are able to outcompete the normal hematopoietic compartments in a subject, thereby leading to hematopoietic failure (in the form of anemia, thrombocytopenia and neutropenia) ultimately causing death. A person of ordinary skill in the art can classify a cancer as a sarcoma, carcinoma or hematopoietic cancer.

Cancer, as used herein, includes the following types of cancer, breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art.

### Therapeutically Effective Amount

In some embodiments, the compounds of the invention can be used in therapeutically effective amounts. The term “therapeutically effective amount” or “effective amount”, which can be used interchangeably, refers to the amount necessary or sufficient to realize a desired therapeutic effect, e.g., shrinkage of a tumor, decrease of angiogenesis, inhibition or suppression of cell proliferation, or increase of the percentage of apoptotic cells in a population of cells. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be planned which does not cause substantial toxicity and yet is effective to treat the particular subject.

The effective amount for any particular application can vary depending on such factors as the disease or condition being treated, the particular piperlongumine or piperlongumine analog being administered, the size of the subject, or the severity of the disease or condition. One of ordinary skill in the art can empirically determine the effective amount of a particular compound of the invention (i.e., piperlongumine or piperlongumine analog) and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug.

In some embodiments, a therapeutically effective amount is less than 50 mg/kg, such as less than 45 mg/kg, less than 40 mg/kg, less than 35 mg/kg, less than 30 mg/kg, less than 25 mg/kg, less than 20 mg/kg or less than 15 mg/kg. In some embodiments, a therapeutically effective amount is less than 10 mg/kg, such as less than 9 mg/kg, less than 8 mg/kg, less than 7 mg/kg, less than 6 mg/kg, less than 5 mg/kg, less than 4 mg/kg, less than 3 mg/kg or less than 2 mg/kg. In some embodiments, a therapeutically effective amount is less than 1.5 mg/kg, such as less than 1.4 mg/kg, less than 1.3 mg/kg, less than 1.2 mg/kg, less than 1.1 mg/kg, less than 1 mg/kg, less than 0.9 mg/kg, less than 0.8 mg/kg, less than 0.7 mg/kg, less than 0.6 mg/kg, less than 0.5 mg/kg, less than 0.4 mg/kg, less than 0.3 mg/kg, less than 0.2 mg/kg or less than 0.1 mg/kg.

In some embodiments, a therapeutically effective amount of a particular piperlongumine or piperlongumine analog is less than the LD50 of that particular piperlongumine or piperlongumine analog, as determined by testing that particular piperlongumine or piperlongumine analog in a model organism, such as mouse, rat or dog, or other disease model. In some embodiments, a therapeutically effective amount of a particular piperlongumine or piperlongumine analog is less than 50%, less than 40%, less than 30%, less than 25%, less than 20%, less than 15%, less than 10%, less than 9%, less than 8%, less than 7%, less than 6%, less than 5%, less than 4%, less than 3% or less than 2% of the LD50 of that particular piperlongumine or piperlongumine analog in a model organism. In some embodiments, a therapeutically effective amount of a particular piperlongumine or piperlongumine analog is less than 1%, less than 0.9%, less than 0.8%, less than 0.7%, less than 0.6%, less than 0.5%, less than 0.4%, less than 0.3%, less than 0.2% or less than 0.1% of the LD50 of that particular piperlongumine or piperlongumine analog in a model organism.

In some embodiments, the therapeutically effective amount is administered in one dose. In some embodiments, the therapeutically effective amount is administered in multiple doses. Dosage may be adjusted appropriately to achieve desired compound levels, local or systemic, depending upon the mode of administration. For example, it is expected that intravenous administration would require a lower dose than oral delivery to result in the same therapeutically effective amount. In the event that the response in a subject is insufficient at such doses, even higher doses (or effective higher doses by a different, more localized delivery route) may be employed to the extent that subject tolerance permits. Multiple doses per day are contemplated to achieve appropriate systemic levels of compounds.

### Pro-Drugs

The invention also embraces the administration of prodrugs of piperlongumine and piperlongumine analogs. The term “prodrug” as used herein refers to any compound that when administered to a biological system generates a biologically active compound (i.e., piperlongumine or a piperlongumine analog) as a result of spontaneous chemical reaction(s), enzyme catalyzed chemical reaction(s), and/or metabolic chemical reaction(s), or a combination of each. Standard prodrugs are formed using groups attached to functionality, e.g. HO—, HS—, HOOC—, R₂N—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Prodrugs can undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described, for example, in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

### Anti-Cancer Compounds

In some embodiments, piperlongumine and/or piperlongumine analogs can be administered combined with other therapeutic agents (Also defined herein as a non-piperlongumine anti-cancer compound). The piperlongumine and/or piperlongumine analogs and other therapeutic agent may be administered simultaneously or sequentially. When the other therapeutic agents are administered simultaneously they can be administered in the same or separate formulations, but are administered at the same time. The other therapeutic agents are administered sequentially with one another and with piperlongumine and/or piperlongumine analogs, when the administration of the other therapeutic agents and the piperlongumine and/or piperlongumine analogs is temporally separated. The separation in time between the administration of these compounds may be a matter of minutes or it may be longer.

In some embodiments, the other therapeutic agent is an anti-cancer compound. As used herein, an “anti-cancer compound” refers to an agent which is administered to a subject for the purpose of treating a cancer. Anti-cancer compounds include, but are not limited to anti-proliferative compounds, anti-neoplastic compounds, anti-cancer supplementary potentiating agents and radioactive agents. One of ordinary skill in the art is familiar with a variety of anti-cancer agents, or can find those agents in the routine art, which are used in the medical arts to treat cancer.

Anti-cancer agents include, but are not limited to, the following sub-classes of compounds: Antineoplastic agents such as: Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Buniodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorombucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Ifesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin, Mitosper; Mitotane; Mitoxantrone Hydrochloride; Mycophenolic Acid; Nocodazole; Nogalamycin; Ormaplatin; Oxisuran; Paclitaxel Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate, Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid, 2-chloro-2′-arabino-fluoro-2′-deoxyadenosine; 2-chloro-2′-deoxyadenosine; anisomycin; trichostatin A; hPRL-G129R; CEP-751; linomide; Piritrexim Isethionate; Sitogluside; Tamsulosin Hydrochloride and Pentomone.

Anti-neoplastic compounds include, but are not limited to 20-epi-1,25 dihydroxyvitamin D3; 5-ethynyluracil; abiraterone; aclarubicin; acylfulvene; adecypenol; adozelesin; aldesleukin; ALL-TK antogonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin 13; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin 10 deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflomithine; elemene; emitefur; epirubicin; epothilones (A, R═H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor, multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; Sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating agents include, but are not limited to, Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca²⁺ antagonists (e.g., verapamil, nifedipine, nitrendipine and caroverine); Calmodulin inhibitor (e.g. prenylamine, trifluoroperazine and clomipramine); Amphotericin B; Triparanol analogues (e.g. tamoxifen); antiarrhythmic drugs (e.g., quinidine); antihypertensive drugs (e.g. reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

Radioactive agents include but are not limited to Fibrinogen I 125; Fludeoxyglucose F18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate-Hg 203; Merisoprol Hg 197; Selenomethionine Se 75; Technetium Tc 99m Atimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium 99m Lidofenin; Technetium Tc 99 mm Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Ic 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125: Thyroxine I 131; Tolpovidone I 131; Triolein I 125; Triolein I 131.

In some embodiments, the compounds of the invention are administered in conjunction with an anti-cancer therapy. Anti-cancer therapies include the administration of anti-cancer compounds, radiation and surgical procedure.

### Pharmaceutical Compositions and Routes of Administration

The compounds of the invention typically are administered as pharmaceutical compositions, which may routinely contain pharmaceutically acceptable concentrations of salt, buffering agents, preservatives, compatible carriers, adjuvants, and optionally other therapeutic ingredients. The nature of the pharmaceutical carrier and other components of the pharmaceutical composition will depend on the mode of administration.

The pharmaceuticals composition of the present invention may be administered by any means and route known to the skilled artisan in carrying out the treatment methods described herein. Preferred routes of administration include but are not limited to oral, parenteral, intratumoral, intramuscular, intranasal, intracranial, sublingual, intratracheal, inhalation, ocular, vaginal, and rectal.

In the course of the experimental investigations described herein, it was found that more than 0.25 mg/ml of piperlongumine in DMSO was precipitated out when diluted 1:10 in phosphate buffered saline or water. To avoid toxicity, piperlongumine should be used at less than 2.5 mg/kg in mice. The skilled person will know how to formulate the compounds of the invention in accordance with the solubility by selection of appropriate carriers, solubilizers, etc.

For oral administration, the compounds of the invention can be formulated readily by combining the compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions, or may be administered without any carriers.

For the compounds of the invention, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection the compound or by release of the biologically active compound beyond the stomach environment, such as in the intestine. To ensure full gastric resistance a coating impermeable to at least pH 5.0 is desired. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

The compounds of the invention can be included in the formulation as fine multi-particulates in the form of granules or pellets of particle size about 1 mm. The formulation of the material for capsule administration could also be as a powder, lightly compressed plugs or even as tablets. The pharmaceutical composition could be prepared by compression. Colorants and flavoring agents may all be included. For example, the compounds of the invention may be formulated (such as by liposome or microsphere encapsulation) and then further contained within an edible product, such as a refrigerated beverage containing colorants and flavoring agents. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the pharmaceutical composition into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the compounds of the invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compounds of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds of the invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the compounds of the invention. The compounds of the invention may delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass. All such devices require the use of formulations suitable for the dispensing the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compounds of the invention may also be prepared in different formulations depending on the type of chemical modification or the type of device employed. Formulations suitable for use with a nebulizer, either jet or ultrasonic, will typically comprise the compounds of the invention dissolved in water at a concentration of about 0.1 to 25 mg of biologically active compound. The formulation may also include a buffer and a simple sugar. The nebulizer formulation may also contain a surfactant, to reduce or prevent surface induced aggregation caused by atomization of the solution in forming the aerosol.

Formulations for use with a metered-dose inhaler device will generally comprise a finely divided powder containing the compounds of the invention suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1, 1, 1, 2-tetrafluoroethane, or combinations thereof. Suitable surfactants include sorbitan trioleate and soya lecithin. Oleic acid may also be useful as a surfactant. Formulations for dispensing from a powder inhaler device will comprise a finely divided dry powder containing the compounds of the invention and may also include a bulking agent, such as lactose, sorbitol, sucrose, or mannitol in amounts which facilitate dispersal of the powder from the device, e.g., 50 to 90% by weight of the formulation. The compounds of the invention should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran. For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available. Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds of the invention, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

The compounds may also be formulated in rectal or vaginal compositions such as suppositories or retention enemas, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference.

The compounds of the invention and optionally other therapeutics, including non-piperlongumine anti-cancer compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of one or more compounds of the invention and optionally additional therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

The compounds of the invention may be provided in particles. Particles as used herein means nano or microparticles (or in some instances larger) which can consist in whole or in part of the compounds of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compounds of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by Sawhney et. al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The compounds of the invention may be contained in controlled release systems. The term “controlled release” is intended to refer to any compound of the invention-containing formulation in which the manner and profile of compound release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound there from. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Kits

In one aspect the invention provides kits comprising a pharmaceutical composition comprising a therapeutically effective amount of piperlongumine and/or piperlongumine analog and instructions for administration of the pharmaceutical composition. In some aspects of the invention, the kit can include a pharmaceutical preparation vial, a pharmaceutical preparation diluent vial, and the compound of the invention. The diluent vial contains a diluent such as physiological saline for diluting what could be a concentrated solution or lyophilized powder of the compound of the invention. In some embodiments, the instructions include instructions for mixing a particular amount of the diluent with a particular amount of the concentrated pharmaceutical preparation, whereby a final formulation for injection or infusion is prepared. In some embodiments, the instructions include instructions for use in a syringe or other administration device. In some embodiments, the instructions include instructions for treating a patient with an effective amount of the compounds of the invention. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

### Identification of Pro-Apoptotic Gene Activators by Small Molecule Library Screening

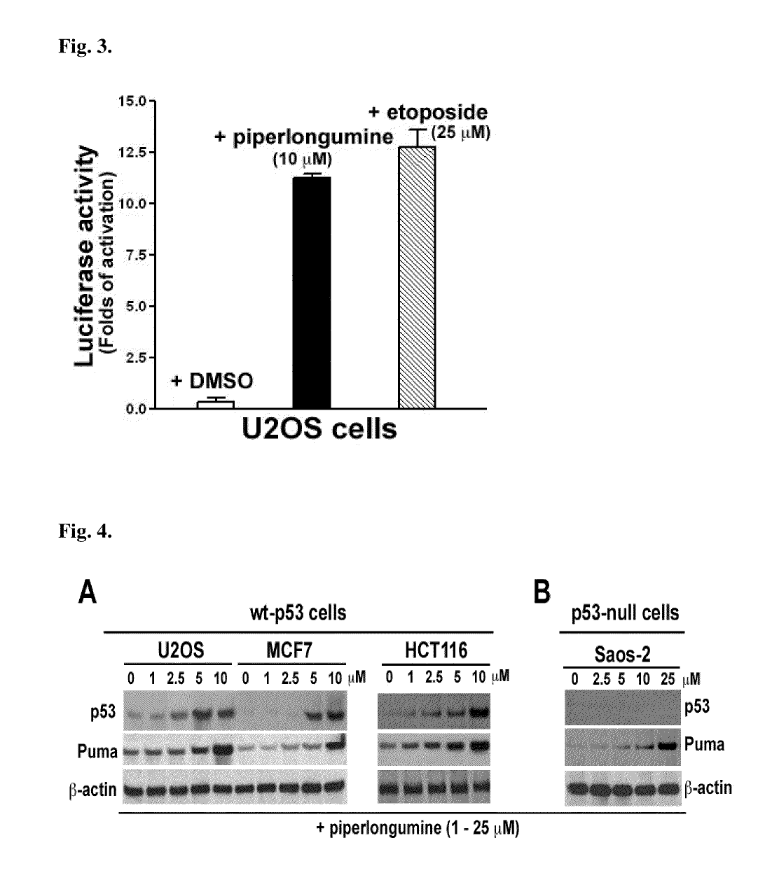

We screened a small molecule library of biologically active compounds using a Luciferase reporter gene construct fused with CDIP (Cell Death Involved p53 target) promoter/p53 responsive site as a read-out assay. p53 transcriptional activators were identified from screening the diversity set of the biologically active library using U2OS human cancer cells expressing the p53-responsive reporter. An overview of the screening process is presented in FIG. 1. Briefly, a reporter construct was created that included the CDIP promoter operatively linked to the luc2 luciferase reporter gene. We stably expressed a human p53 reporter, luciferase2/Puro+CDIP promoter, which carries the firefly luciferase gene under the control of p53-responsive elements of CDIP promoter, in U2OS cells. Stable cell lines were produced that expressed the reporter construct, and the cells were plated in 384 well plates at ˜10,000 cells per well. Test compounds were added to the plated cells at two replicates per compound, and the cells were incubated with the compounds for 24 hours. The data were analyzed using Spotfire. The initial screen identified several compounds that activated p53-responsive reporter expression during 48 hours. Among a number of candidates the natural compound with the highest composite Z value was piperlongumine (FIG. 2). As shown in FIG. 3, piperlongumine treatment (10 μM) significantly increased luciferase activity of the CDIP promoter containing p53 binding site in U2OS cells.

### Example 2

### Induction of p53 Target Gene Expression by Piperlongumine

Western blot analyses showed that p53 expression was significantly induced by using relatively low concentrations of piperlongumine in different types of cells including U2OS and HCT116 human cancer cells (FIG. 4). We also found that the expression of tumor suppressor p53 was significantly increased by piperlongumine treatment. Moreover, other p53 proapoptotic targets such as Puma (p53-upregulated modulator of apoptosis) was also significantly induced in response to piperlongumine. Thus, piperlongumine is a p53 activator as well as an activator for proapoptotic targets.

### Example 3

### Piperlongumine is Effective in Killing Human Cancer Cells In Vitro

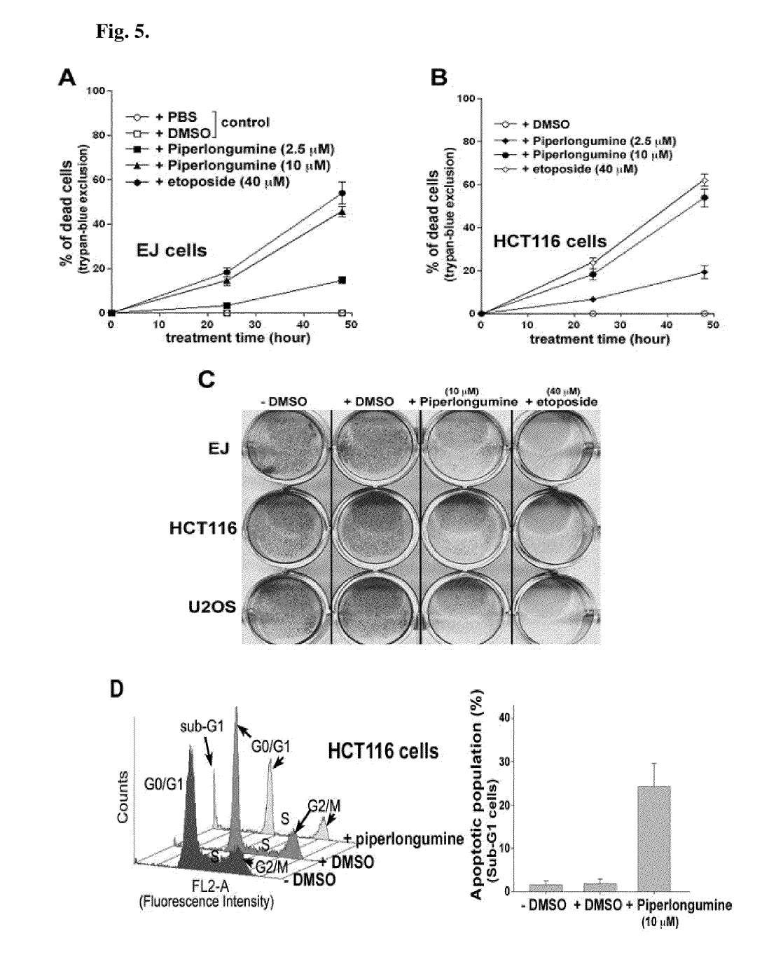

We evaluated the ability of piperlongumine to induce apoptosis in a panel of human cancer cells (>40 human cancer cell lines) including cell lines with both wt and mutant p53 status, including non-functional mutants (See Table 1). We found that piperlongumine inhibited tumor cell growth and induced cell death/apoptosis in various human cancer cells including breast cancer cells, bladder cancer cells, colon cancer cells, ovarian cancer cells, lung cancer cells, melanoma and prostate cancer cells at micromolar potencies (2.5-20 microM), regardless of p53 status (FIG. 5).

TABLE 1
Human cancer cell lines and tumor suppressor p53 status

| Cell line | Tissue of origin/tumor type | p53 status |
|---|---|---|
| MCF7 | Breast | wt-p53 |
| HCT116 | Colon | wt-p53 |
| U2OS | Osteosarcoma | wt-p53 |
| EJ | Bladder | mutant (unfunctional) |
| Saos-2 | Osteosarcoma | p53-null |
| A-549 | Lung cancer | wt-p53 |
| DLD1 | Colon | mutant |
| SW620 | Colon | mutant |
| CAKI-1 | Renal | mutant |
| M19-MEL | Melanoma | mutant |
| M14 | Melanoma | mutant |
| SK-OV-3 | Ovarian | mutant |

### Example 4

### In Vivo Anti-Tumor Effects of Piperlongumine (CT-007)

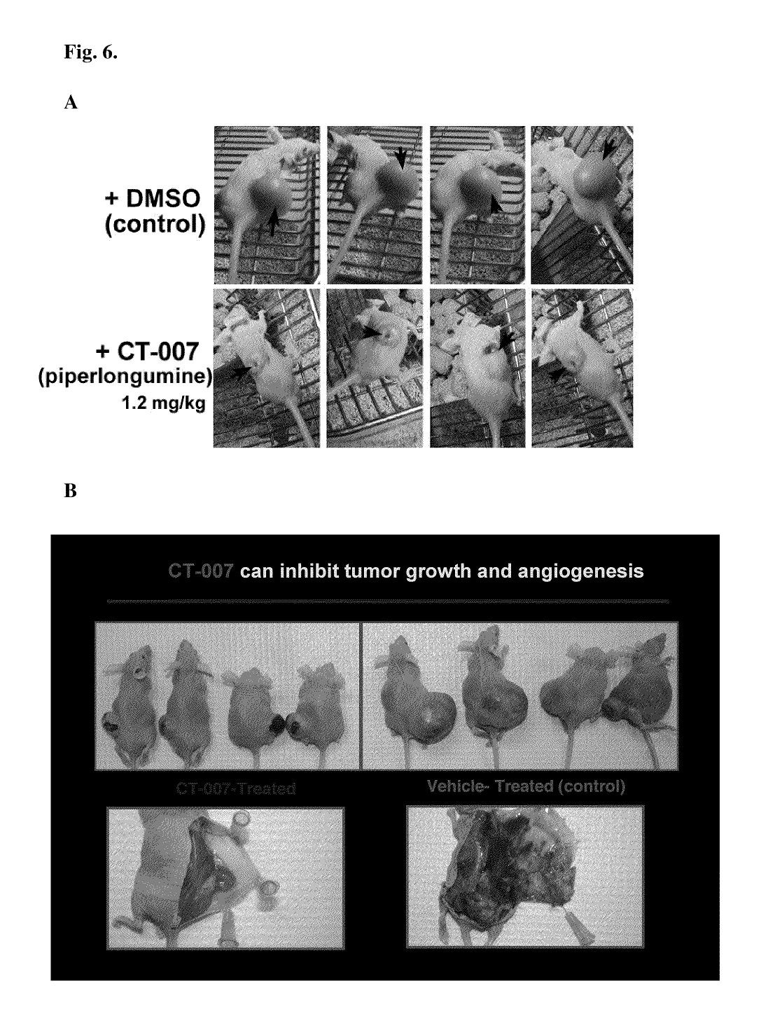

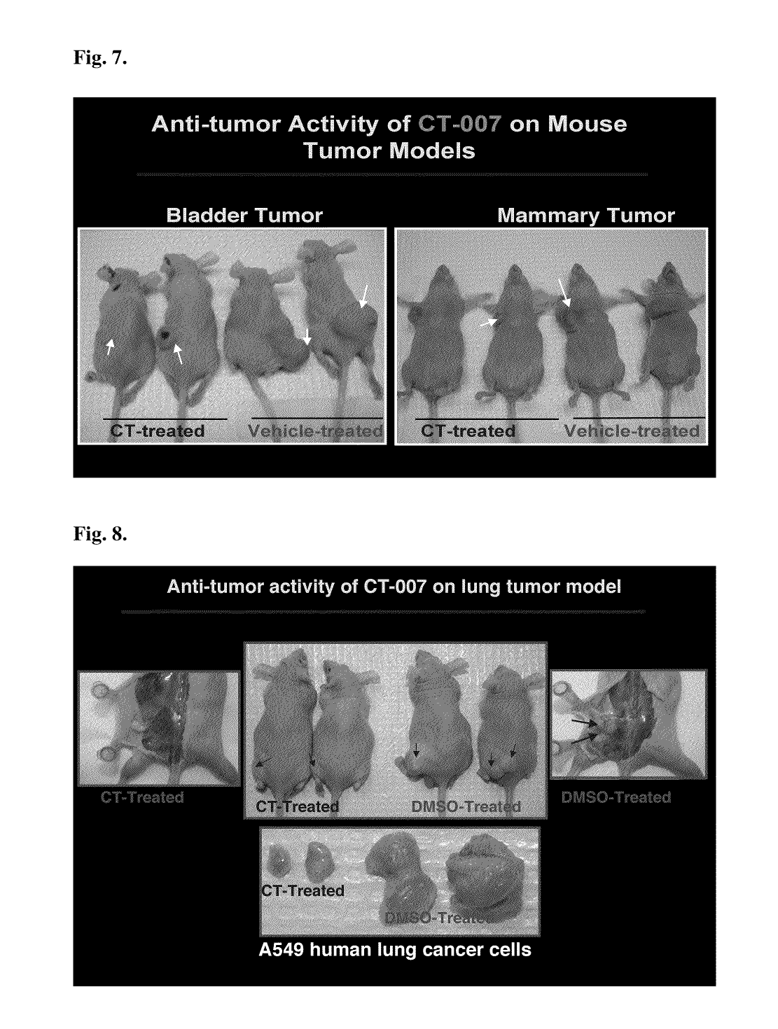

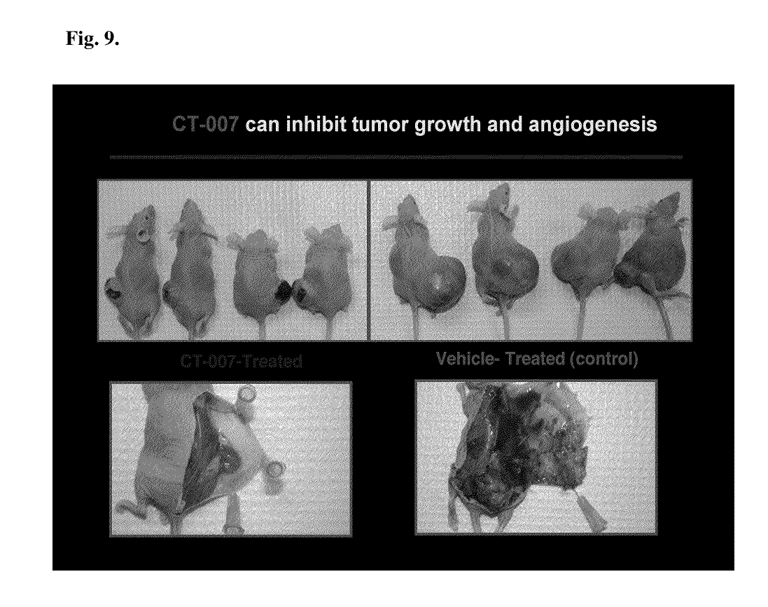

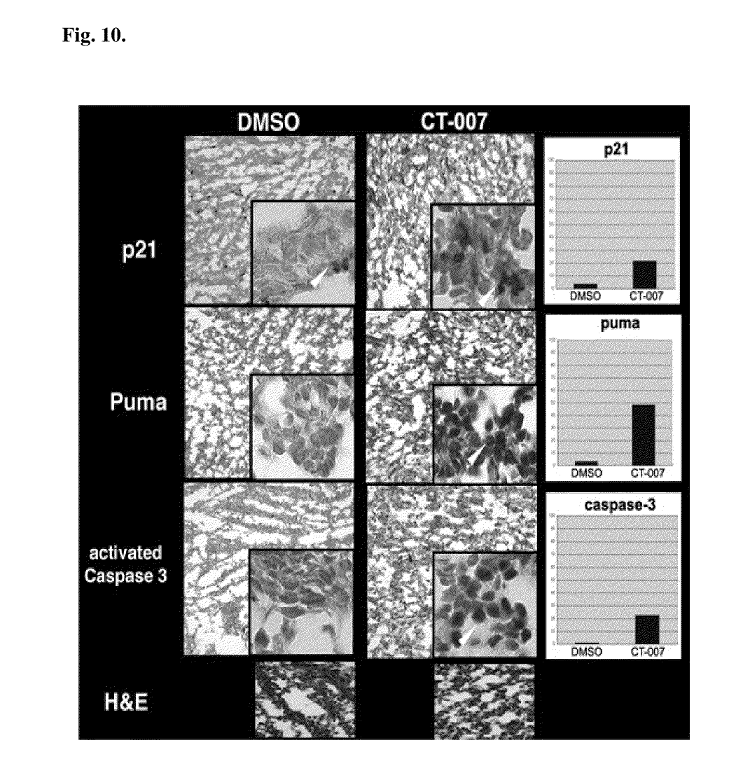

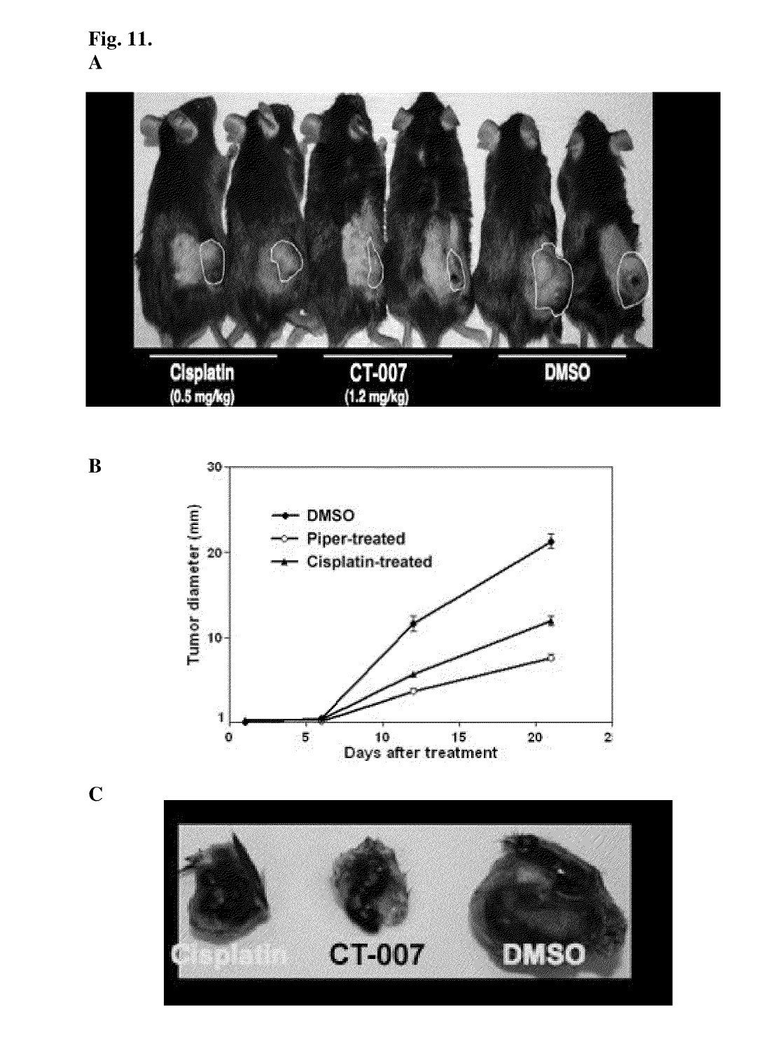

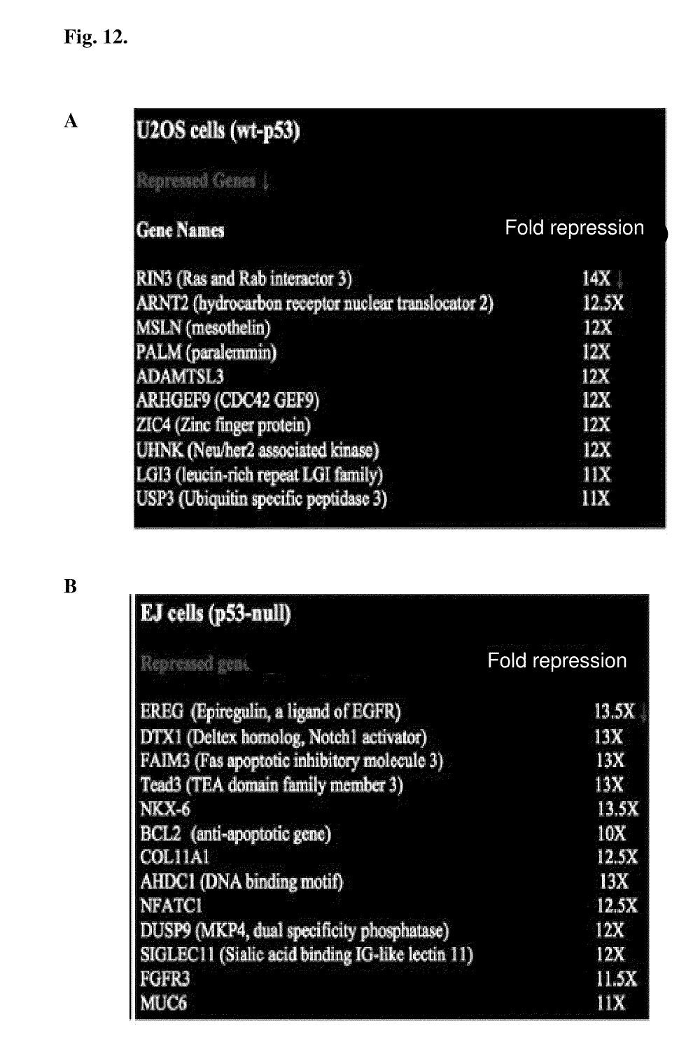

We tested piperlongumine in colon tumor, breast, melanoma or bladder tumor xenograft-bearing mice to evaluate the anti-tumor effect of piperlongumine. We examined EJ human bladder cancer cell xenografts (non-functional p53) to evaluate anti-tumor effects. A total of 2×10⁶ EJ cells or SW480 cells were implanted subcutaneously on opposite site flanks in each of six nude/nude mice in each group. When tumor masses grew to ˜5-10 mm in diameter, piperlongumine was administered intraperitoneally (28 ug per each intraperitoneal administration, total 1.2 mg/kg) every 48 hours for 12 days (6 times total). As shown in FIG. 6, significant anti-tumor effects were observed in piperlongumine (CT-007) administered tumor mice, as compared to control DMSO-administered tumor mice. FIGS. 7 and 8 also show the effective killing by piperlongumine (CT-007) of breast cancer and lung cancer tumors, respectively, in mice. Moreover, FIG. 9 shows that piperlongumine (CT-007) treatment strongly inhibited blood vessel formation (angiogenesis) in the tumors. We also found that piperlongumine (CT-007) treatment enhanced expression of apoptosis genes in tumor-mice, including p21, PUMA (p53-upregulated modulator of apoptosis) and Caspase 3 (FIG. 10). The results clearly show that treatment with piperlongumine hindered tumor growth.

### Example 5

### In Vivo Anti-Tumor Effects of Piperlongumine (CT-007) in Wild Type Mice

We tested the effectiveness of piperlongumine in wild type mice using mouse B16-F10 melanoma cells. A total 2×10⁶ cells were implanted subcutaneously on opposite site flanks in each of 12 B6 wild type mice in each group. When tumor masses grew to ˜5-10 mm in diameter, piperlongumine was administered intraperitoneally (28 ug per each intraperitoneal administration, total 1.2 mg/kg) every 48 hours for 12 days (6 times total). As shown in FIG. 11, significant anti-tumor effects were observed in piperlongumine-administered tumor mice, as compared to control DMSO-administered tumor mice.

### Example 6

### Downstream Targets of Piperlongumine (CT-007) in Cancer Cells

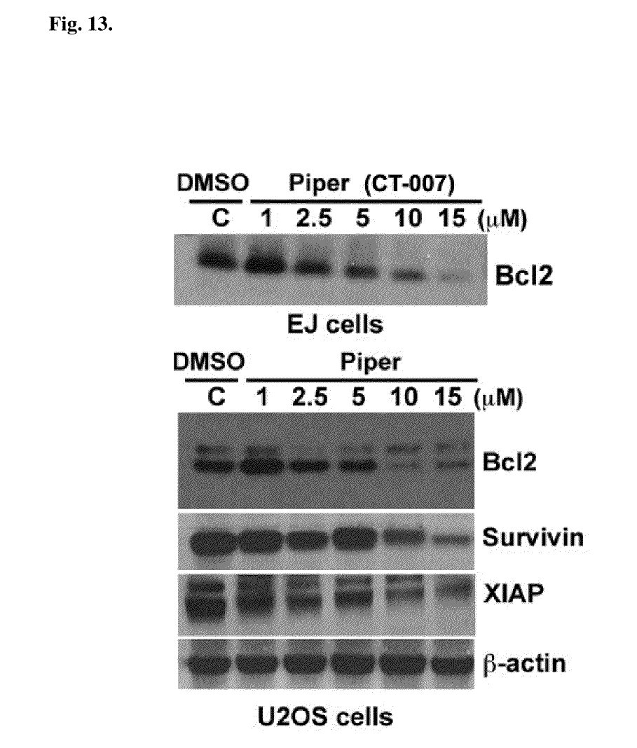

Downstream target genes of piperlongumine (CT-007) were identified using an human exon gene array analysis of U2OS and EJ cancer cell lines (FIG. 12). In addition, the results from the gene array analysis were confirmed by Western blot (FIG. 13; See also Example 12 below). The experiments reveal that piperlongumine treatments significantly repressed the levels of expression of several important survival proteins in cancer cells.

### Example 7

### Related Compounds Show No Anti-Tumor Activity

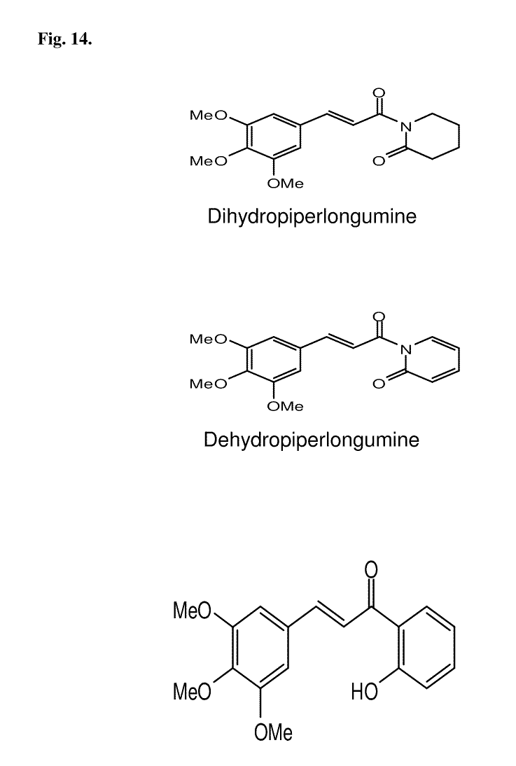

FIG. 14 shows compounds related to piperlongumine that were tested for anti-tumor activity. None of the compounds showed any anti-tumor activity. The experimental details are the same as in Example 4. In addition, the compounds related to piperlongumine were also not able to induce p53. The experimental details are the same as in Example 8 below.

### Example 8

### Induction of p53 Acetylation by Piperlongumine

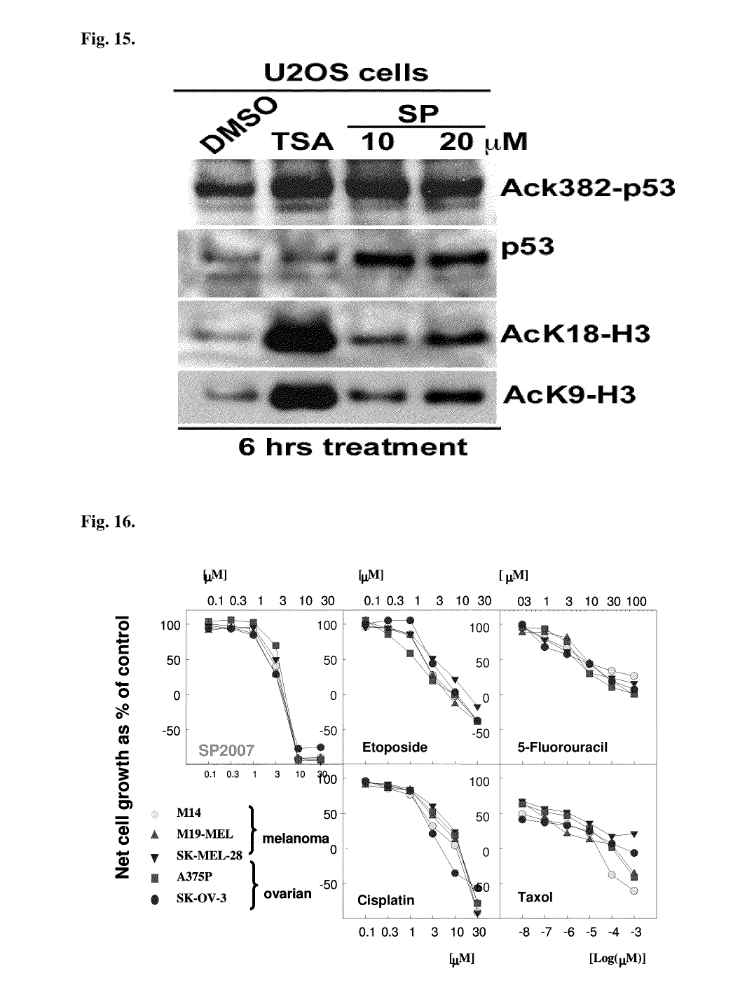

U2OS cells were treated with piperlongumine (SP), the HDAC inhibitor TSA (trichostatin A (10 mM)) or the control DMSO for 6 hours. Cell extracts were fractionated in SDS-PAGE gel and analyzed by western blot. Piperlongumine treatment increased the amount of acetylated p53 to a level similar to TSA treated cells. However, treatment with TSA did not increase the total amount of p53 while treatment with piperlongumine did result in an increased in the total amount of p53 (FIG. 15).

### Example 9

### Inhibition of Cell Growth of Cancer Cell Lines by Piperlongumine

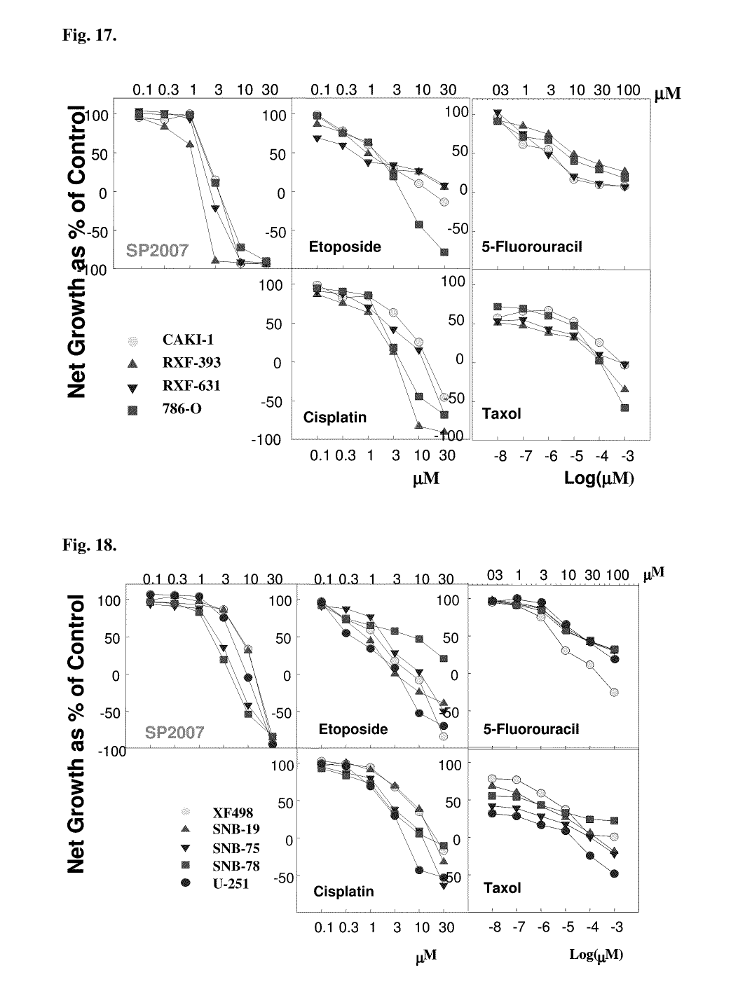

The tumor growth inhibitory effect of piperlongumine was evaluated in a variety of human cancer cell lines. Cells were grown in ˜50-70% confluency and treated with piperlongumine and known anti-cancer agents at various concentrations (0.1-30 μM) and analyzed for cell death or viability by SRB (Sulforhodamine B) staining assay. The SRB assay is a well established assay that is used for the detection of cell death, viability or proliferation in drug screening. Piperlongumine inhibits tumor cell growth as well as or better than other well-known anti-cancer drugs such as etoposide and taxol. FIG. 16 shows the treatment of human melanoma and ovarian cancer cell lines. FIG. 17 shows the treatment of human renal cancer cell lines. FIG. 18 shows the treatment of glioblastoma cell lines.

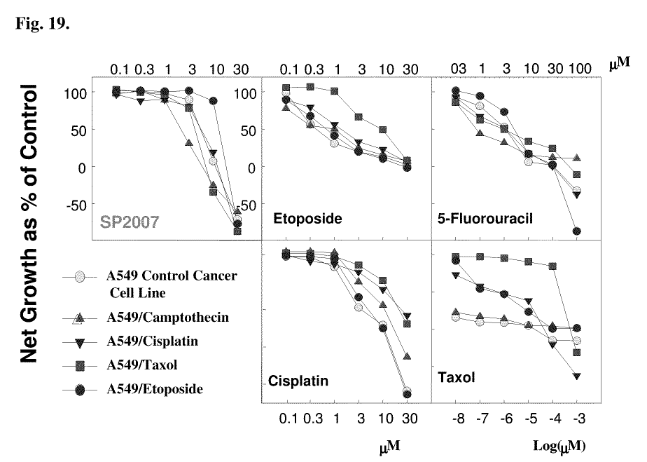

Tumor cell killing by piperlongumine was also tested in drug-resistant A549 lung cancer cell lines and compared to known anti-cancer agents (see FIG. 19). Piperlongumine was also more effective in inhibiting tumor cell growth than other anti-cancer drugs in drug-resistant A549 tumor cell lines.

### Example 10

### Piperlongumine Induces Cell Death in Transformed Cells but not in Control Cells

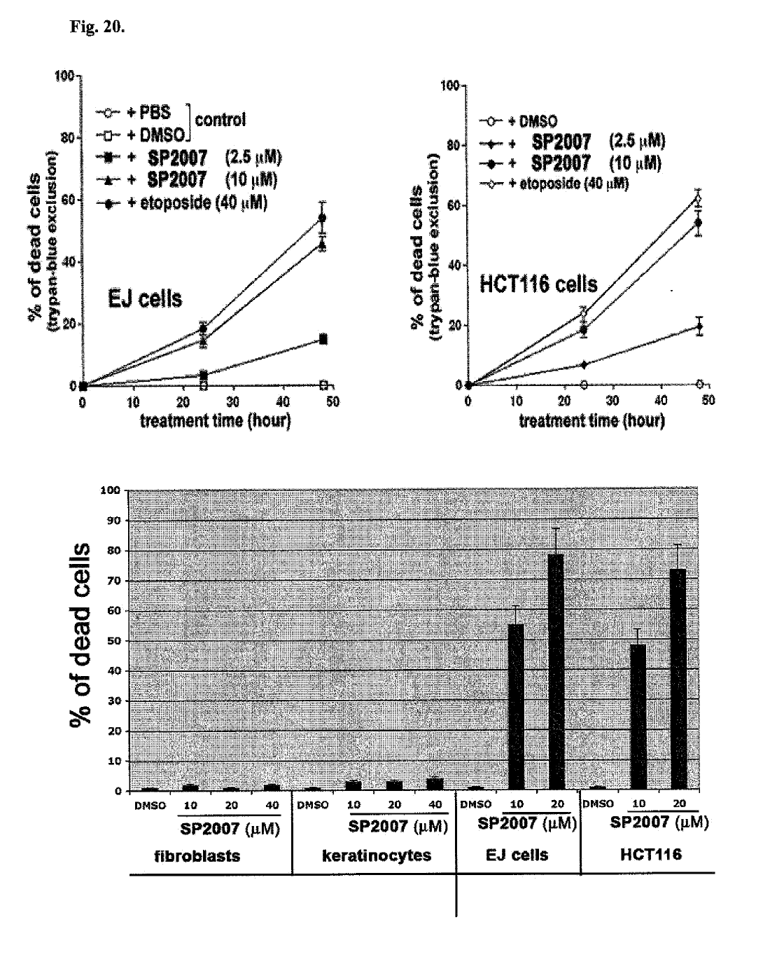

Transformed cancer cells (EJ bladder carcinoma cells and HCT116 colon carcinoma cells) and non-transformed control cells (diploid fibroblasts, keratinocytes and breast epithelial cells) were treated with piperlongumine and the known anti-cancer compound etoposide. Piperlongumine induced cell death in transformed cancer cells at relatively low concentrations (2.5 μM and 10 μM) but etoposide did not induce cell death in control cells even at a concentration of 40 μM (FIG. 20).

### Example 11

### Piperlongumine Changes the miRNA Profile of Cancer Cells

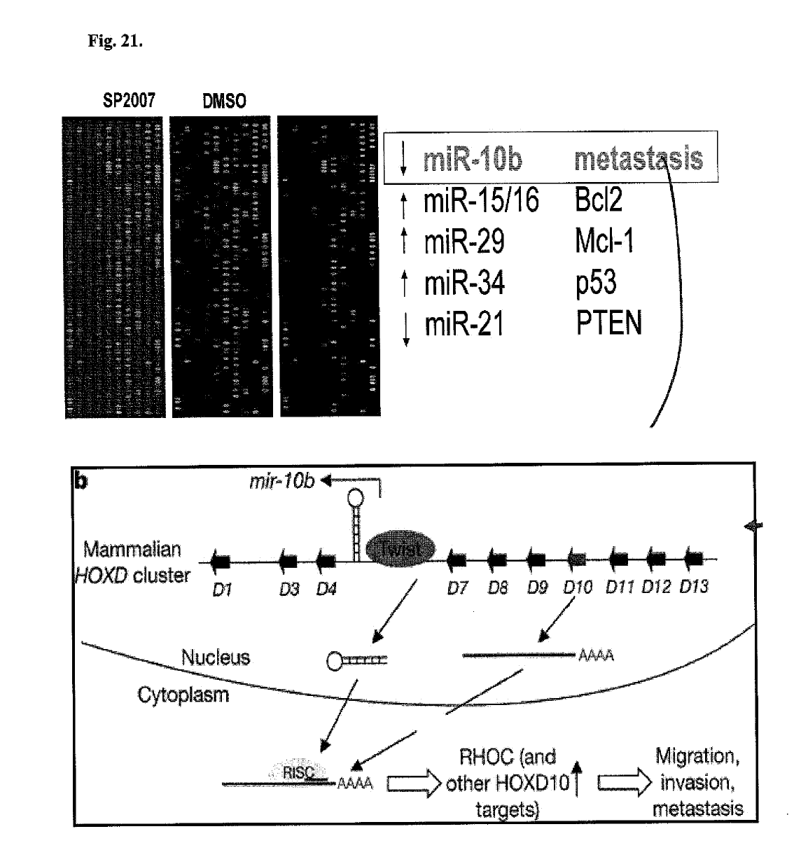

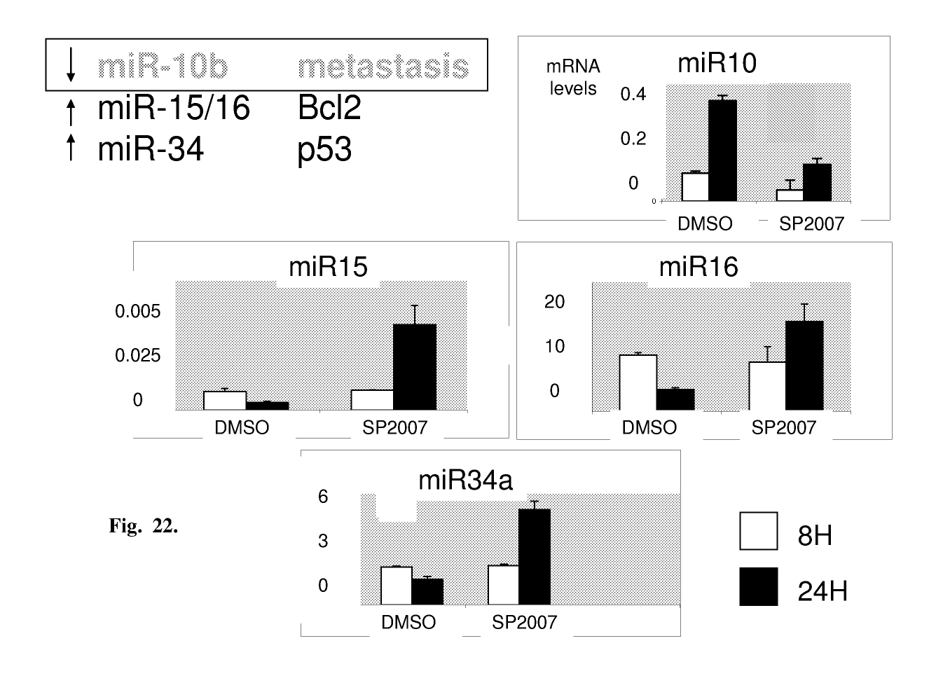

U2OS cells and EJ cells were treated with piperlongumine (10 mM) and total RNA was extracted after 0, 6 and 24 hrs using miRNeasy Mini Kit (Qiagen, Germantown, Md.) according to the manufacturer's instructions. The miRNA microarray analysis was done by LC Sciences (Houston, Tex.). Total RNA (10 μg) was size fractionated (<200 nucleotides) by using a mirVana kit (Ambion, Austin, Tex.) and labeled with Cy3 or Cy5 fluorescent dyes. Dye switching was done to eliminate the dye bias. Pairs of labeled samples were hybridized to dual-channel microarrays. Microarray assays were done on a μParaFlo microfluidics chip with each of the detection probes containing a nucleotide sequence of coding segment complementary to a specific miRNA sequence and a long non-nucleotide molecule spacer that extended the detection probe away from the substrate. miRNA detection signal threshold was defined as twice the maximum background signal. The maximum signal level of background probes was 180. Normalization was done using a cyclic LOWESS (locally weighted regression) method to remove the system-related variations. Data adjustments included data filtering, log2 transformation, and gene centering and normalization (Analysis performed at LCScience). The t-test analysis was conducted between treated and non-treated and U2OS and EJ samples, and miRNA with P values<0.05 were selected for cluster analysis. The clustering analysis was done using a hierarchical method and average linkage and Euclidean distance metrics. miRNA-10b is a target for TWIST (FIGS. 21 and 22).

### Example 12

### Inhibition of Twist and Induction of CDIP and Other Survival Proteins by Piperlongumine

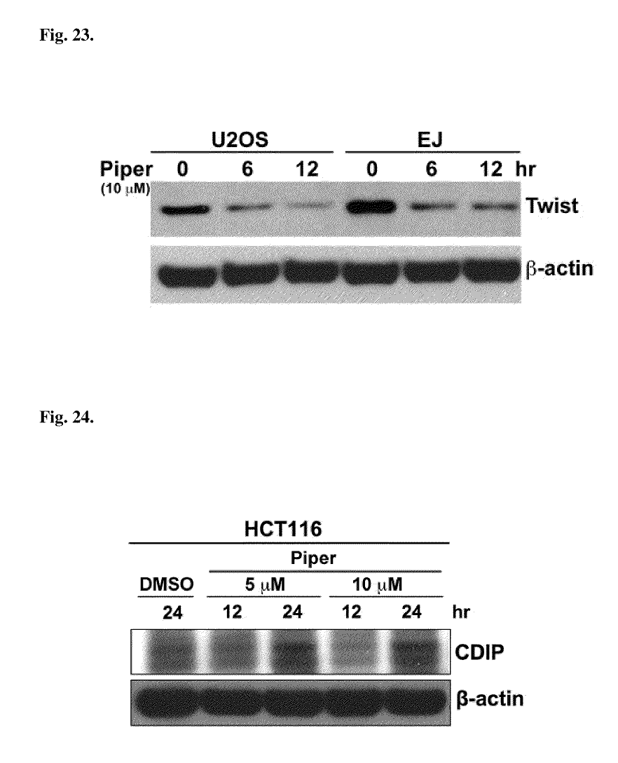

U2OS cells and EJ cells were exposed to piperlongumine (10 μM) and aliquots of the cells were taken at 6 and 12 hours and lysed to determine the level of Twist expression. FIG. 23 shows that piperlongumine (piper) inhibits the expression of Twist. HCT116 cells (p53-wt cells) were exposed to piperlongumine and aliquots of the cells were taken at 6 and 12 hours and lysed to determine the level of CDIP expression. FIG. 24 shows that piperlongumine induces the expression of CDIP at both 5 μM and 10 μM concentration. U2OS cells (p53-wt cells) and EJ cells (non-functional p53) were exposed to various concentrations of piperlongumine (piper) and aliquots of the cells were lysed to determine the level of survival protein expression. FIG. 13 shows that piperlongumine inhibits the expression of survival proteins (Bc12, Survivin, XAIP) in human bladder cancer cells (EJ cells with non-functional p53) as well as in human osteosarcoma U2OS cells with wt-p53 at a variety of concentrations. β-actin was used as a loading control in all experiments.

### Example 13

### Piperlongumine Inhibits Cancer Cell Growth in Patient Derived Samples

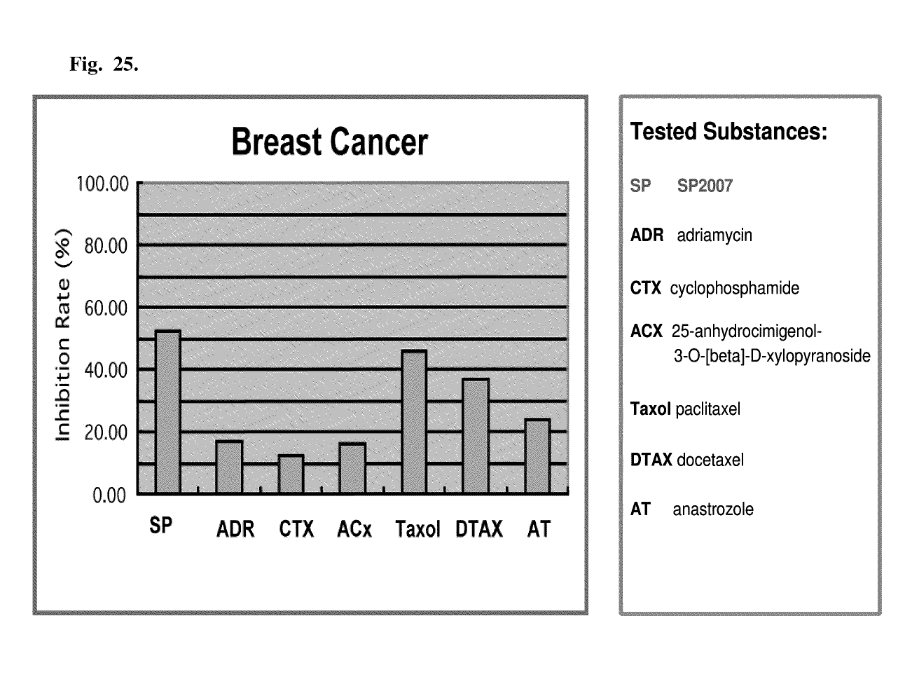

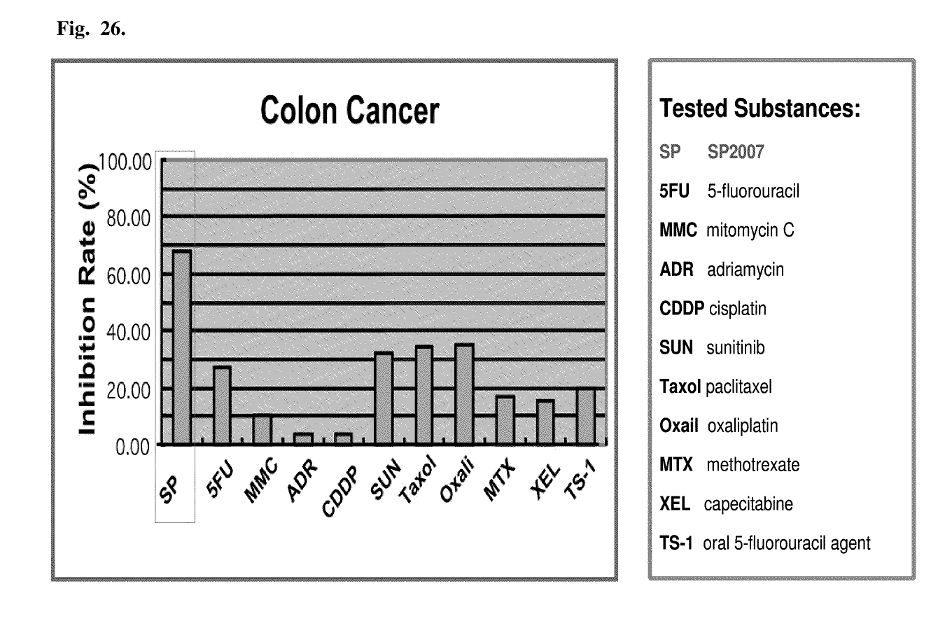

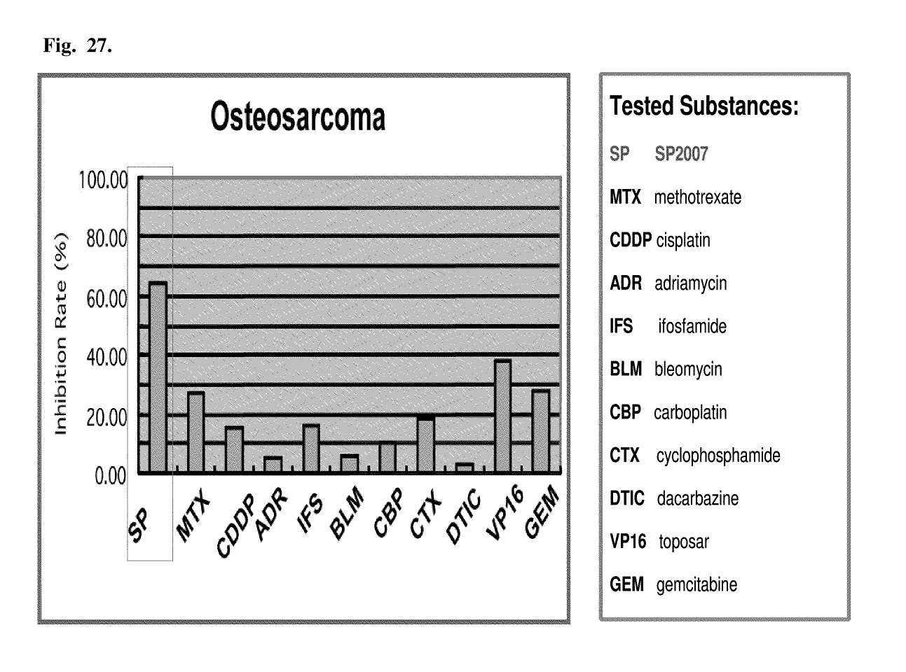

Piperlongumine (SP2007) was compared to a variety of known anti-cancer agents in a Histoculture Drug Response Assay (HDRA). HDRA is an assay that provides a reliable approach to determine the profile of in vivo chemo-sensitivity in cancer patients. The assay allows for a three-dimensional culture, the testing of patient tumor sensitivity, the evaluation of treatment option for individual patients with solid tumor types and a high correlation to clinical drug sensitivity (Flowers J L et al., Cancer Chemother. Pharmacol. 2003 September 52 (3): 253-261). The assays were performed by Anti-cancer Inc., (San Diego, Calif.). An average of twenty samples was tested for each tumor type. FIG. 25 shows the results for patient-derived breast cancer cells. FIG. 26 shows the results for patient-derived colon cancer cells. FIG. 27 shows the results for patient-derived breast osteosarcoma.

### Example 14

### Piperlongumine Suppresses Angiogenesis

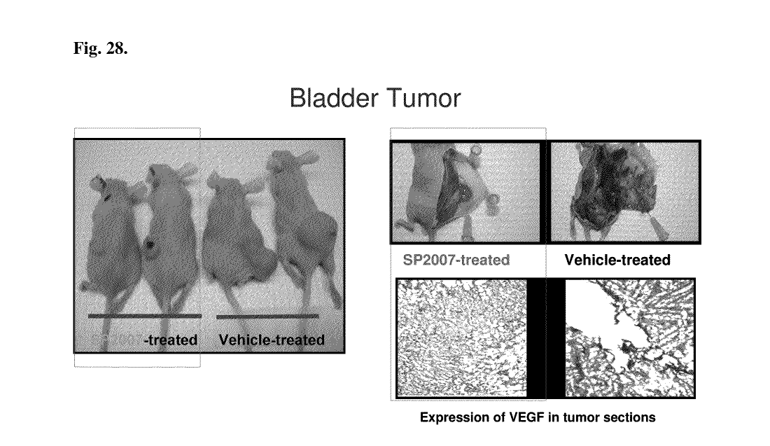

EJ Human Bladder Carcinoma Cells (10⁶ Cells) were Subcutaneously Transplanted into the back of the skin of immunodeficient nude mice (total 16 mice). After 2 weeks (tumor sizes of approximately 8-10 mm), piperlongumine (SP2007; 1.5 mg/kg) or vehicle (10% DMSO) was injected into tumor bearing mice by i.p. every two days for 2.5 weeks. The effect of piperlongumine was evaluated by opening the tumor site. Vehicle-treated tumor mice showed a highly angiogenic structure, but piperlongumine-treated tumor mice (SP2007) did not show considerable blood-vessel formation at the tumor sites. In addition, immunohistochemistry with an anti-VEGF antibody showed that the expression of an angiogenic marker VEGF was significantly reduced in piperlongumine-treated tumor mice, as compared to vehicle-treated tumor mice (FIG. 28).

### Example 15

### Piperlongumine Suppresses Tumor/Progression and Migration

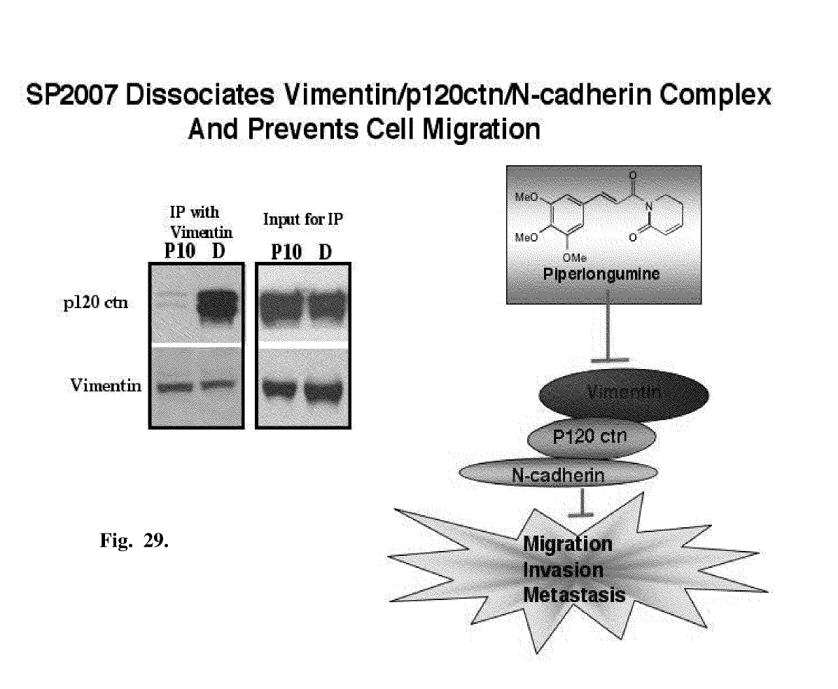

We previously showed that Twist expression was inhibited by piperlongumine. It is well-established that Twist plays a major role in tumor development and metastasis. Thus, we investigated whether piperlongumine (SP2007) mechanistically regulates a key signaling pathway involving a complex of p120ctn/vimentin/N-cadherin which is a Twist target that promotes tumor cell migration and invasion/metastasis. EJ cells were treated with 10 μM piperlongumine or vehicle for 20 hours and cell lysates were subsequently immunoprecipitated by Vimentin antibody (Sigma Aldrich, St. Louis, Mo.) followed by western blot against p120ctn to analyze the association of the p120ctn/vimentin/N-cadherin complex. The data presented in FIG. 29 show that piperlongumine treatment of EJ cells results in the dissociation of the p120ctn/vimentin/N-cadherin complex, implying that piperlongumine suppresses tumor progression and migration through the inhibition of p120ctn/vimentin/N-cadherin complex formation.

### Example 16

### Long-Term Piperlongumine Treatment Shows No Toxic Effects

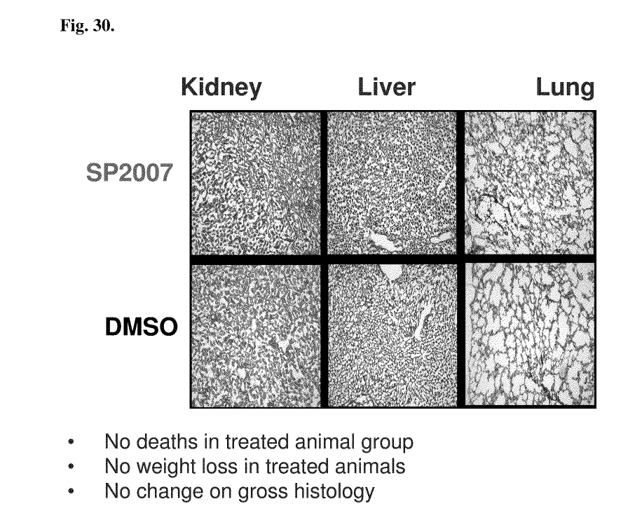

Piperlongumine (2.4 mg/kg) was injected into mice (8 mice per group) multiple times for 4 weeks. None of the mice died. After 4 weeks the mice were killed and examined for changes in gross histology in target organs. FIG. 30 shows that no change in gross histology in kidney, liver and lung was observed as assessed by hematoxilin and eosin staining of frozen sections of the different tissues.

### Example 17

### Piperlongumine Inhibits Tumor Progression in a Spontaneous Tumor Model

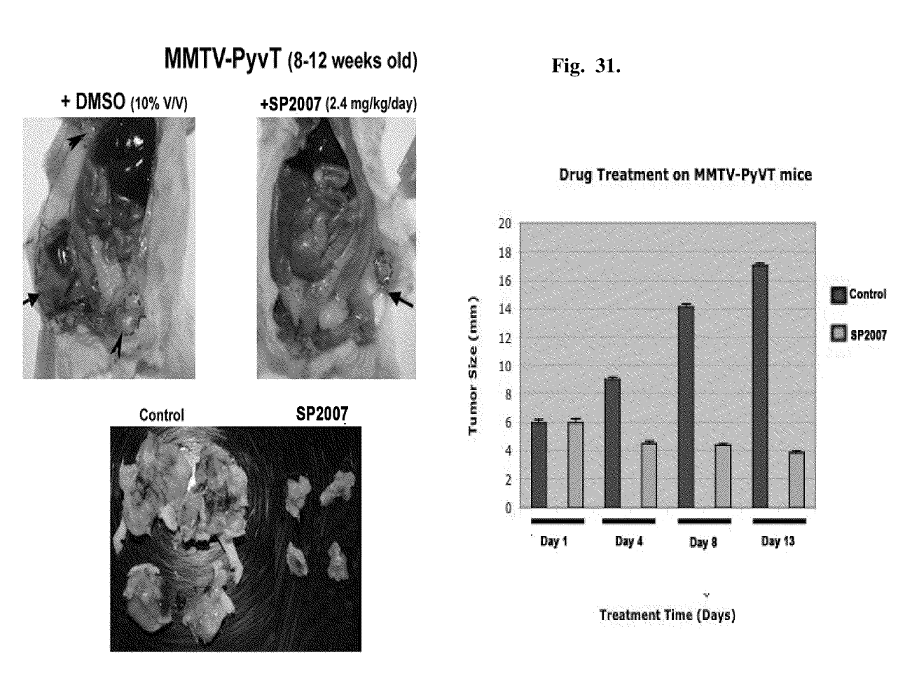

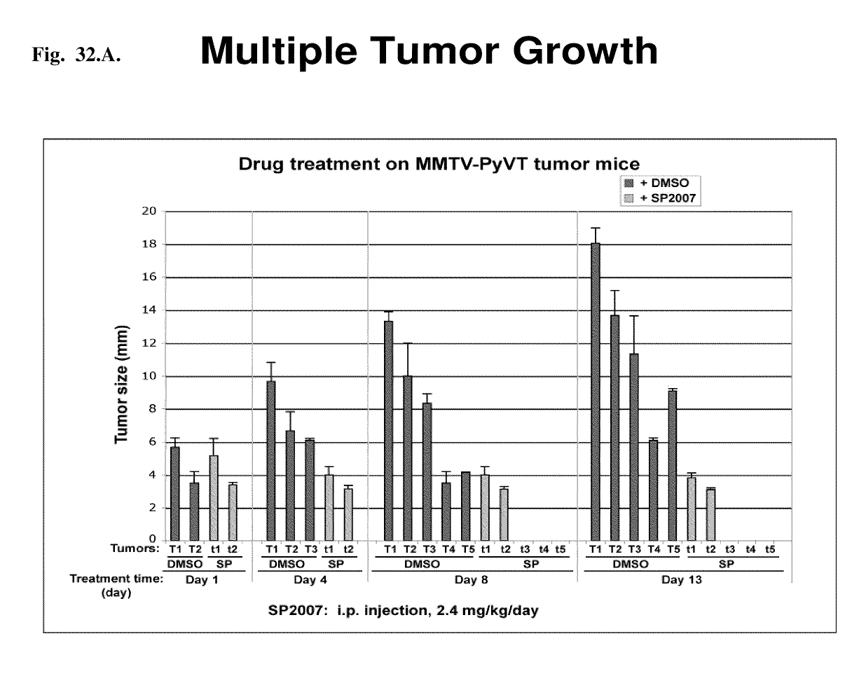

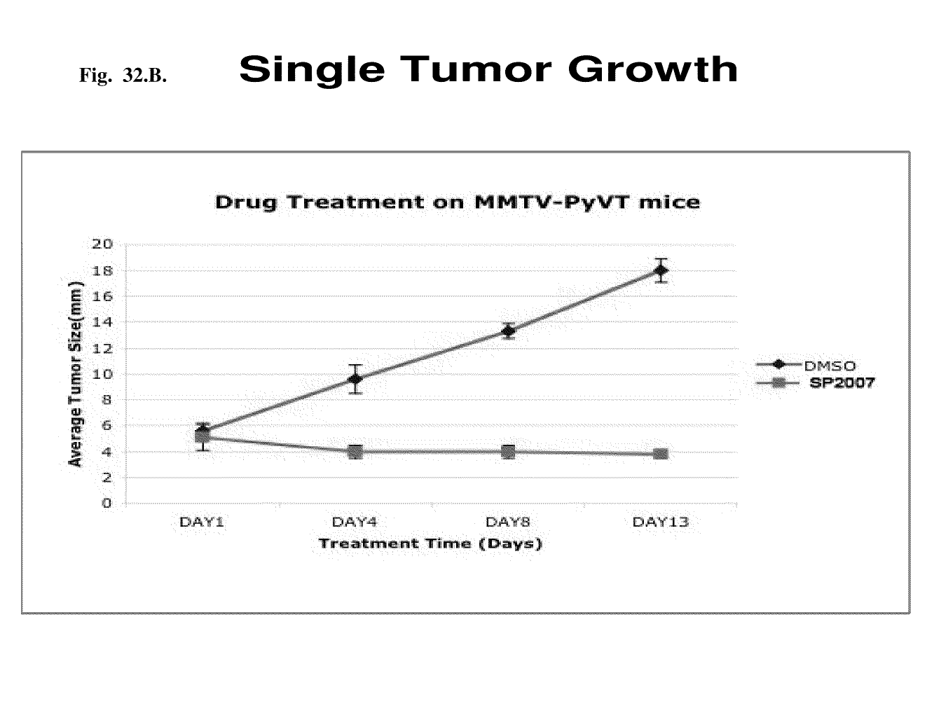

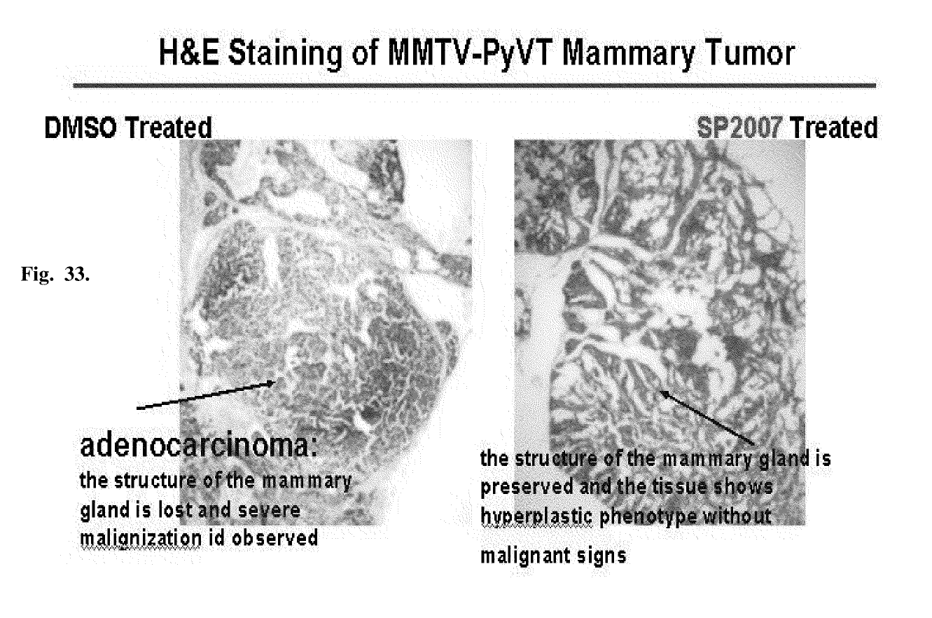

MMTV-PvVT (FVB/N-Tg) transgenic mice develop multifocal mammary tumor (from hyperplasia to metastatic) with a high incidence of metastasis (Mice were obtained from the NCI repository). This mouse model has been widely used for the correlation studies of human cancer (Guy et al., Moll. Cell. Biol. 12: 954-961, 1992). In addition, this onco-mouse model has been used to evaluate the effectiveness of potential drug therapies. Generally, due to aggressiveness of tumor growth and spread in this mouse-model, only a combination of chemo-drugs is effective in tumor growth inhibition. MMTV-PyVT mice were maintained until mammary tumor size reached to 6 mm, and then piperlongumine (2.4 mg/kg) or vehicle (DMSO) was given daily by i.p. for 13 days. Tumor size was measured every 4 days (See FIGS. 31 and 32). FIG. 33 shows the immunohistochemistry staining of tumor samples in vehicle and piperlongumine treated mice.

### Example 18

### Piperlongumine Analogs Inhibit Cell Proliferation and Induce Puma and p53

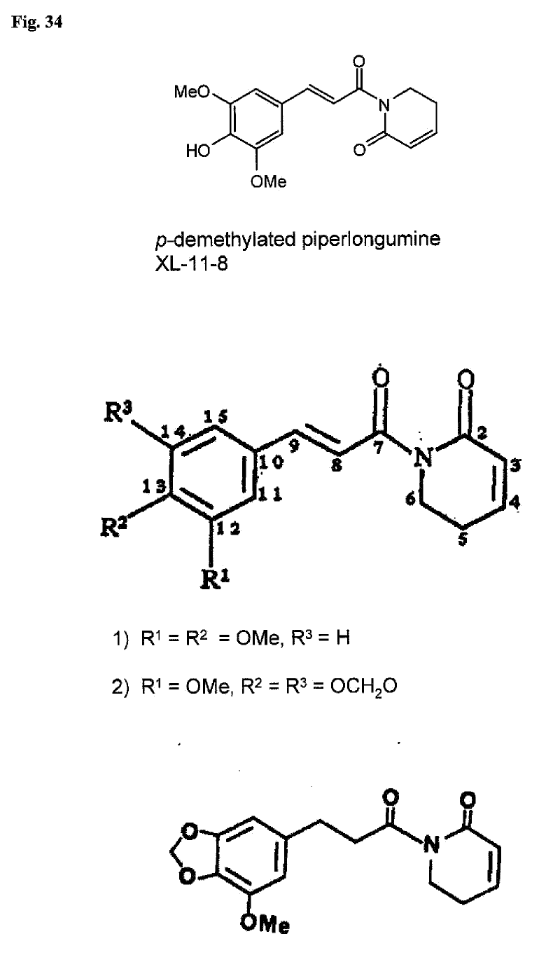

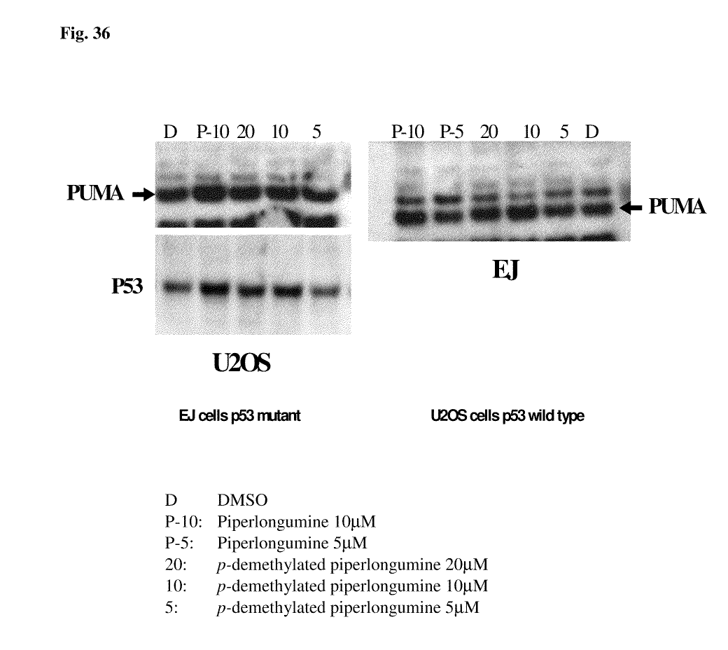

EJ cells and U2OS cells were treated with piperlongumine and the piperlongumine analog XL-11-8 (p-demethylated piperlongumine) at 10 μM and 20 μM concentration (See FIG. 34). FIG. 35 shows that the piperlongumine analog XL-11-8 inhibits proliferation of EJ cells and U2OS cells better than piperlongumine at the same concentration. The y-axis of the graphs of FIG. 35 indicate the percentage of dead cells 12 hrs after treatment with the compound. FIG. 36 shows that the piperlongumine analog XL-11-8 induces the expression of PUMA and p53.

### Example 19

### Mammary Tumor Growth Inhibition by SP2007 or Taxol Treatment in Breast Transgenic Tumor Mice

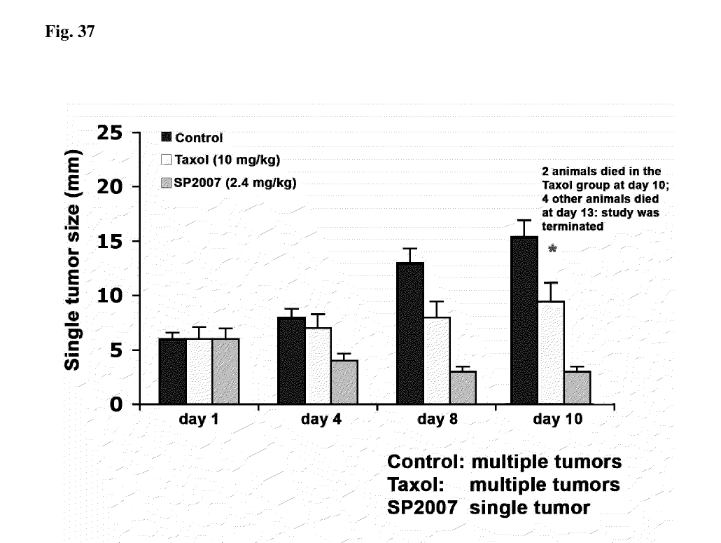

We compared the anti-tumor activity of piperlongumine (SP2007) with taxol, a well known chemo drug, in the MMTV-PyVT tumor mouse model as described above (8 mice per group). When tumor sizes grew to ˜5-6 mm in diameter, SP2007 (2.4 mg/kg/day) or Taxol (Paclitaxel, 10 mg/kg/day) was administered intraperitoneally (I.P.) daily for two weeks. After two weeks treatment, mice were sacrificed, and mammary tumors excised and the tumor sizes measured. SP2007-treated mice remained healthy throughout the treatment time. The size of the grossly dissected tumors was measured using the formula: mean diameter=(A+B)/2. Significant anti-tumor effects were observed in SP2007-administered MMTV-PyVT mice, as compared to control DMSO-administered MMTV-PyVT mice. In contrast, two of the Taxol-treated mice died at day 10 and moderate and secondary tumors appeared. No secondary tumors were observed in SP2007-treated mice (See also FIG. 37).

### Example 20

### SP2007 Treatment Induces CDIP in U2OS Human Cancer Cells Containing wt-p53

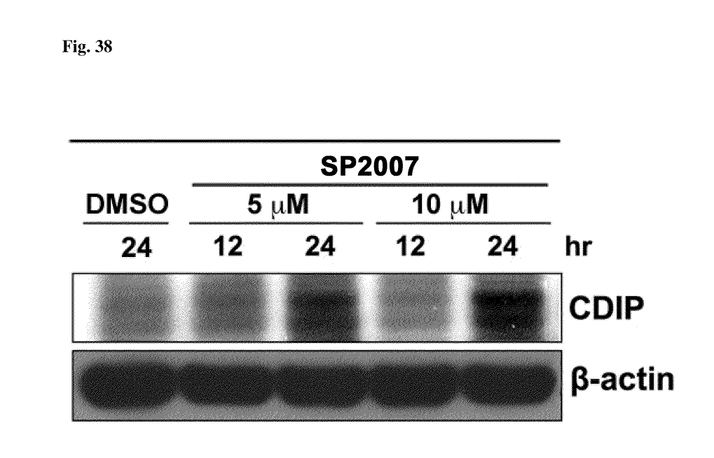

Western blot analyses of CDIP expression in human cancer cells containing wt-p53 treated with piperlongumine (SP2007) (5 μM and 10 μM) is shown in FIG. 38. Beta-actin expression was used as a loading control.

### Example 21

### SP2007 Inhibits Expression of Twist and N-Cadherin in Cancer Cells

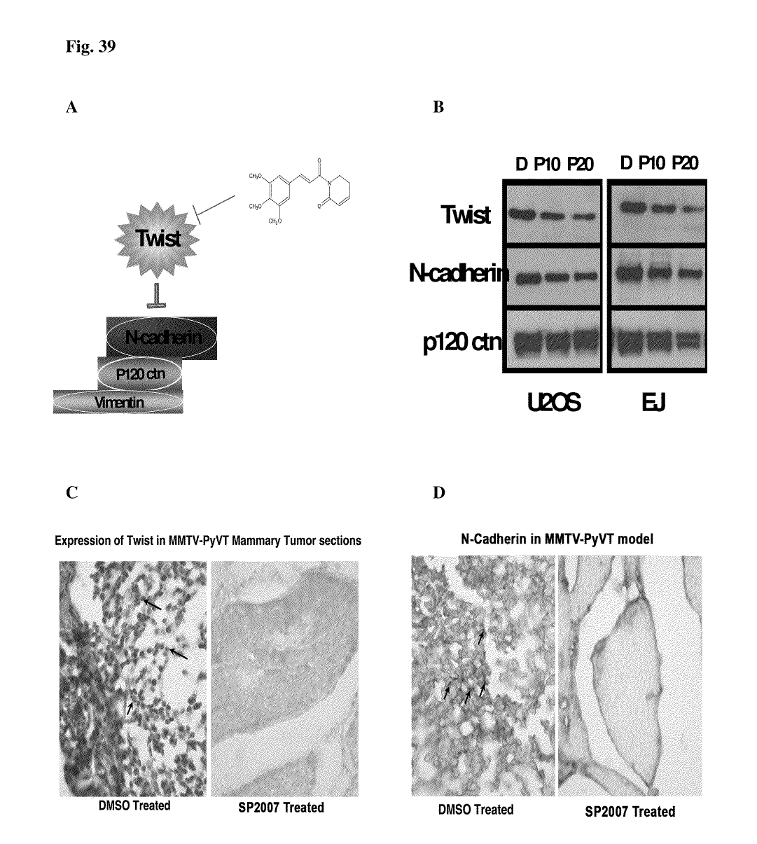

A scheme for piperlongumine (SP2007)-mediated repression of Twist expression and its downstream targets that are involved in tumor invasion/metastasis is presented in FIG. 39A. FIG. 39B shows Western blot data that indicate that SP2007 inhibits expression of Twist and its targets N-cadherin and p120 catenin in EJ and U2OS human cancer cells. U2OS and EJ cells were treated with SP2007 at two concentrations (10 μM and 20 μM) as well as with DMSO as a solvent control. After 20 hours, cell lysates were extracted and western blotting was performed against Twist, N-cadherin and p120 catenin (Zymed and Sigma Aldrich). FIG. 39C shows that SP2007 treatment inhibits Twist expression in MMTV-PyVT mammary tumor mice. Female MMTV-PyVT mice at 8-9 weeks age were chosen for the studies. When tumor sizes grew to ˜5-6 mm in diameter, SP2007 or DMSO was administered intraperitoneally (I.P., total 2.4 mg/kg of SP2007) daily for two weeks. After two weeks treatment, mice were sacrificed and mammary tumors excised and processed for histological examination. FIG. 39D shows that SP2007 treatment inhibits N-cadherin expression in MMTV-PyVT mammary tumor mice.

### Example 22

### SP2007 Treatment Disrupts the p120-CTN Complex with Vimentin in EJ Cancer Cells

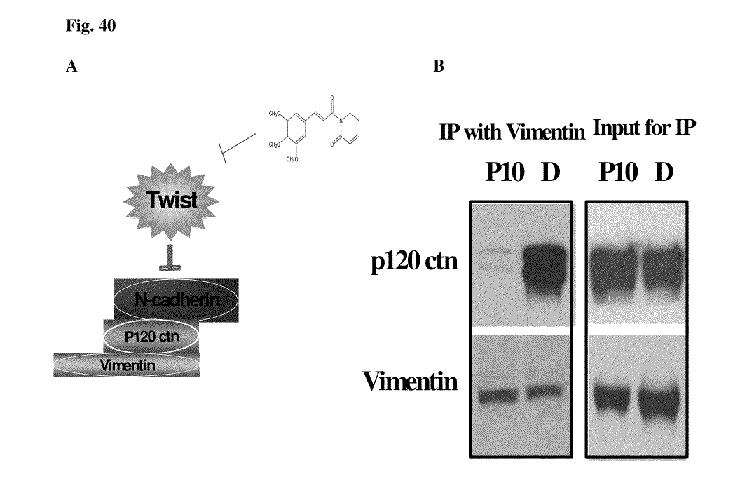

EJ cells were treated with piperlongumine (SP2007) (10 μM) for 12 hours. Subsequently the cells were harvested and subjected to immunoprecipitations using vimentin antibodies (Sigma Aldrich) to precipitate the vimentin complex. Western blots were performed for p120 CTN and vimentin. DMSO (D) treated cells were used as control IP experiments. The right panel in FIG. 40B shows an input immunoblot. In SP2007 treated (P10) cells, p120 was not in the complex precipitated with vimetin, while in DMSO-treated cells p120 was precipitated with vimentin, implying that SP2007 inhibited the complex formation between p120 and vimentin, which plays a critical role in tumor progression and invasion/metastasis (Hsu et al., Cancer Res. 2007, 22: 11064)

### Example 23

### SP2007 induces DNA Damage Selectively in Cancer Cells but not in Normal Human Epithelial Cells

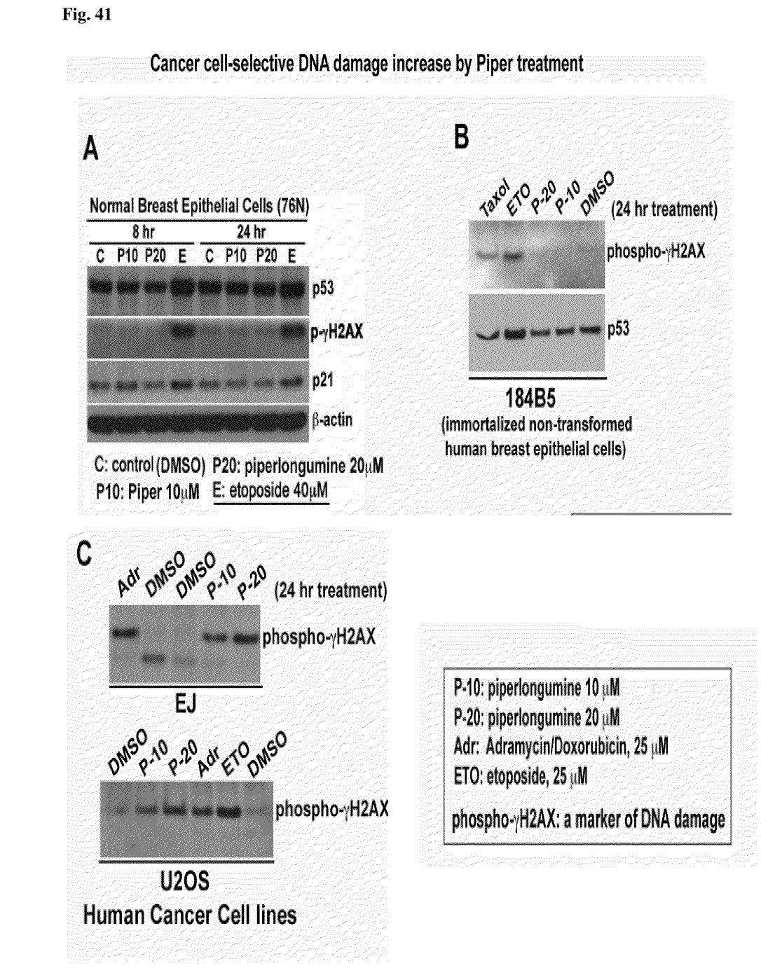

FIG. 41A shows that treatment of normal human breast epithelial cells with DNA damaging agent etoposide treatment induces p53 and p21 as well as a DNA damage marker phosphorylated gamma-H2AX. In contrast, SP2007 (10 μM or 20 μM) does not affect phosphorylated gamma-H2AX, p53 and p21 in the same cells The conditions and time of exposure are indicated in the legend of the figure. The primary breast epithelial cells obtained from patients undergoing reconstructive plastic surgery.

FIG. 41B shows that treatment of immortalized human breast epithelial cells (equivalent of the hyperplastic pre-cancerous cells in vivo) with taxol (10 nM) and etoposide results in the induction of phosphorylated gamma-H2AX and p53. In contrast, treatment with piperlongumine (SP2007) does not result in the induction of phosphorylated gamma-H2AX or p53 in the same cells. The conditions and time of exposure are indicated in the legend of the figure. FIGS. 41C shows that the treatment of both EJ bladder carcinoma and U2OS osteosarcoma cell lines with either etoposide, adrimycin or SP2007 results in the induction of DNA damage (as evidenced by the induction of phosphorylated gamma-H2AX levels).

### Example 24

### Measuring DNA Damage

In order to further confirm that SP2007 specifically induces DNA damage in cancer cells only, we will determine the levels of DNA damage in both cancer and non-cancer cells by Comet assays (For instance by using Trevigen's Comet single cell gel electrophoresis assay). A Comet Assay is a single cell gel electrophoresis assay that provides a simple and effective method for evaluating DNA damage in cells. The assay is based on the ability of denatured, cleaved DNA fragments to migrate out of the cell under the influence of an electric field, whereas undamaged DNA, which migrates slower, remains within the confines of the nucleoid when a current is applied. Evaluation of the DNA “comet” tail shape and migration pattern allows for assessment of DNA damage in a cell. In short, cells are immobilized in a bed of low melting point agarose on a Comet Slide. Following a gentle cell lysis, samples are treated with alkali to unwind and denature the DNA and hydrolyze sites of damage. The samples are then submitted to electrophoresis and staining with a fluorescent DNA intercalating dye. The sample is then visualized by epifluorescence microscopy.

### Example 25

### The Persistent Effects of Piperlongumine (SP2007) after Removal of the Compound

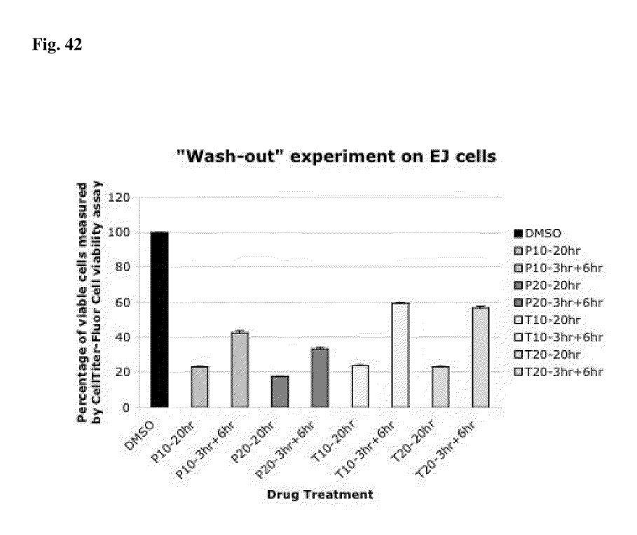

EJ bladder carcinoma cells were treated with SP2007 (P10: 10 μM and P20: 20 μM), taxol (T10: 10 nM and T20: 20 nM) or DMSO vehicle control for either 20 hrs or 3 hrs. For the 20 hrs experiment, the percentages of remaining living cells ware measured on the end of the 20 hrs treatment time point. No difference was observed between Taxol and SP007. For the 3 hrs experiment, cells were washed 3 times with PBS after treatment with the and incubated for another 6 hrs in growing medium without SP2007, taxol or DMSO. Measurement of the percentage of living cells on the end of these 6 hrs showed that the killing effects of SP2007 was still able to kill cancer cells, while the capacity of taxol to kill cancer cells was significantly weakened (FIG. 42).

### Example 26

### Clearance Studies of Piperlongumine In Vivo

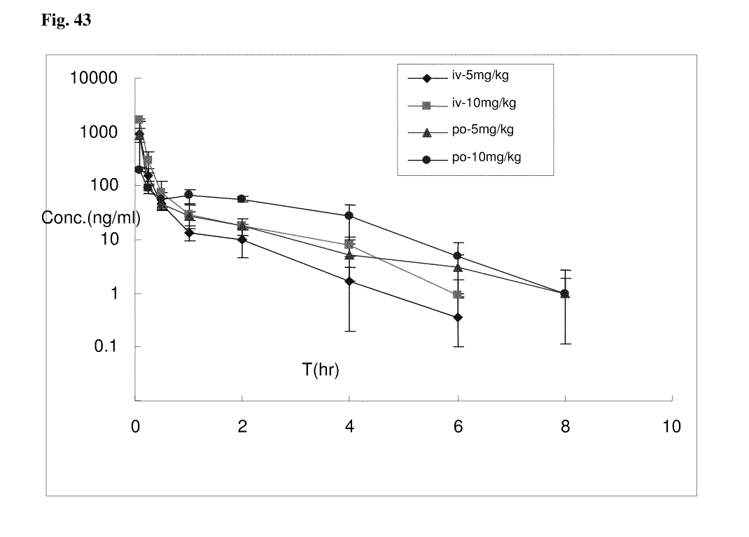

Piperlongumine was administered to C57BL/6 mice at 5 mg/kr or 10 mg/kg both intravenously and orally. FIG. 43 shows the plasma concentration-time curve of piperlongumine in C57BL/6 mice following intravenous (iv) and oral (po) administration (mean±SD, n=3).

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety, particularly for the use or subject matter referenced herein.

We claim:

1. A method for treating a cancer in a subject, the method comprising:
administering to a subject in need of such treatment a therapeutically effective amount of a composition comprising piperlongumine and/or a piperlongumine analog to treat the cancer in the subject.

2. (canceled)

3. (canceled)

4. The method of claim 1, wherein the cancer is a carcinoma, a sarcoma or a melanoma.

5. (canceled)

6. (canceled)

7. The method of claim 1, wherein the piperlongumine analog comprises a piperlongumine conformation.

8. The method of claim 1, wherein the piperlongumine analog is a piperlongumine compound in which one or more methoxy groups are replaced with a hydroxy group.

9. The method of claim 1, wherein the piperlongumine analog is p-demethylated piperlongumine.

10. The method of claim 1, wherein the effective amount is less than 50 mg/kg of piperlongumine or piperlongumine analog.

11. (canceled)

12. (canceled)

13. (canceled)

14. (canceled)

15. (canceled)

16. (canceled)

17. (canceled)

18. (canceled)

19. (canceled)

20. A method for reducing angiogenesis in a subject, the method comprising: administering to a subject in need of such treatment a therapeutically effective amount of a composition comprising piperlongumine and/or a piperlongumine analog to reduce angiogenesis in the subject.

21. A method for reducing metastasis and/or invasion of a cancer in a subject, the method comprising:
administering to a subject in need of such treatment a therapeutically effective amount of a composition comprising piperlongumine and/or a piperlongumine analog to reduce metastasis and/or invasion of the cancer in the subject.

22. A method for increasing apoptosis of a cell or in a population of cells, the method comprising:
contacting the cell or population of cells with an effective amount of a composition comprising piperlongumine and/or a piperlongumine analog to increase apoptosis of the cell or in the population of cells.

23. (canceled)

24. (canceled)

25. (canceled)

26. A method for increasing p53 activity in a cell or population of cells, the method comprising:
contacting the cell or population of cells with an effective amount of a composition comprising piperlongumine and/or a piperlongumine analog to increase p53 activity in the cell or population of cells.

27. (canceled)

28. (canceled)

29. (canceled)

30. The method of claim 26, wherein the cell or population of cells is in a subject.

31. A method for inducing DNA damage in a cancer cell or population of cancer cells, the method comprising:
contacting the cancer cell or population of cancer cells with an effective amount of a composition comprising piperlongumine and/or a piperlongumine analog to induce DNA damage in the cancer cell or population of cancer cells.

32. The method of claim 31, wherein the cancer cell or population of cancer cells is in a subject.

33. A method for preferentially inducing DNA damage in a cancer cell or population of cancer cells, the method comprising:
contacting the cancer cell or population of cancer cells with an effective amount of a composition comprising piperlongumine and/or a piperlongumine analog to induce DNA damage in the cancer cell or population of cancer cells,
wherein the cancer cell or population of cancer cells is in a mixed population of cancer cells and normal cells.

34. The method of claim 33, wherein the mixed population of cancer cells and normal cells is in a subject.

35. A method for suppressing DNA damage in a cell or population of cells, the method comprising:
contacting the cell or population of cells with an effective amount of a composition comprising piperlongumine and/or a piperlongumine analog to suppress DNA damage in the cell or population of cells.

36. (canceled)

37. (canceled)

38. The method of claim 35, wherein the cell or population of cells is in a subject.

39. A pharmaceutical composition comprising piperlongumine and/or a piperlongumine analog and a pharmaceutically acceptable carrier.

40. (canceled)

41. A kit comprising a pharmaceutical composition comprising a therapeutically effective amount of piperlongumine and/or a piperlongumine analog, and instructions for preparation and/or administration of the pharmaceutical composition.

42. (canceled)

43. (canceled)

44. The method of claim 22, wherein the cell or population of cells is in a subject.
